(19) United States
(12) Patent Application Publication
(10) Pub. No.: US 2009/0241202 A1
(43) Pub. Date: Sep. 24, 2009

(54) DOMAIN-GRAFTED ANTIBODIES

(75) Inventors: Peter Kufer, Moosburg (DE); Tobias Raum, München (DE); Petra Lutterbüse, München (DE); Bernd Schlereth, Germering (DE); Klaus Brischwein, München (DE); Robert Hofmeister, Scituate, MA (US)

Correspondence Address: FULBRIGHT & JAWORSKI L.L.P., 600 CONGRESS AVE., SUITE 2400, AUSTIN, TX, 78701, US

(73) Assignee: MICROMET AG, Munchen (DE)

(21) Appl. No.: 12/095,822

(22) PCT Filed: Dec. 14, 2006
(86) PCT No.: PCT/EP2006/012078

§ 371 Date: Dec. 18, 2008

(30) Foreign Application Priority Data
Dec. 15, 2005 (EP) 05027530.4

(51) Int. Cl. A61K 49/00; C07K 16/00; C12N 15/63; C12N 5/00; C12P 21/00

(52) U.S. Cl. 800/3; 530/387.3; 435/320.1; 435/325; 435/69.6

(57) ABSTRACT

The present invention relates to antibodies. Specifically, the present invention relates to antibodies composed of novel combinations of inter-species antibody domains (“domain-grafted antibodies”). The present invention further relates to compositions comprising such domain-grafted antibodies, as well as methods for producing such domain-grafted antibodies. Finally, the present invention relates to methods of evaluating the in vivo biological effect of such domain-grafted antibodies as well as uses of such domain-grafted antibodies for cross-species evaluation of the in vivo activity antibodies intended for human therapy.

The present invention relates to antibodies. Specifically, the present invention relates to antibodies with novel combinations of inter-species antibody regions, or domains (“domain-grafted antibodies”). The present invention further relates to compositions comprising such domain-grafted antibodies, as well as methods for producing such domain-grafted antibodies. Finally, the present invention relates to methods of evaluating the in vivo biological effect of such domain-grafted antibodies as well as uses of such domain-grafted antibodies for cross-species evaluation of the in vivo activity of antibodies intended for human therapy.

Monoclonal antibodies play an increasingly important role in the therapy of many human diseases such as cancer. Most often, the monoclonal antibodies employed in such therapies are of the IgG1 isotype, i.e. monoclonal antibodies comprising the gammal subclass of heavy chain, as described i.a. in Table 4-1 of “Kuby Immunology” Fourth Edition; Richard A. Goldsby, Thomas J. Kindt and Barbara A. Osborne; Published by W.H. Freeman publishers, 2002. A schematic representation of an IgG1 antibody is shown in FIG. 1 of the present application. An IgG1 antibody is composed of four polypeptide chains, two of which are antibody heavy chains, and the other two of which are antibody light chains. Each heavy chain is composed of, from the N-terminus to the C-terminus: a heavy chain variable region (VH), a first heavy chain constant region (C_{H}1), a hinge region, a second heavy chain constant region (C_{H}2) and a third heavy constant region (C_{H}3). Each light chain is composed of, from the N-terminus to the C-terminus: a light chain variable region (VL) and a light chain constant region (C_{L}). As is known in the art, the combination of the VH and VL regions (shaded in FIG. 1) allows the antibody to recognize and bind to molecules, or antigens, while the constant regions, especially the hinge, C_{H}2 and C_{H}3 regions are responsible for triggering certain effector functions. In FIG. 1, inter-chain disulfide bonds are indicated as dotted lines (intra-chain disulfide bonds are not depicted), while locations of glycosylation within the constant regions are indicated as small hexagons. In an antibody of the IgG1 type, these glycosylation sites are located in the C_{H}2 region. As shown in FIG. 1, one heavy chain is disulfide-bonded to one light chain, and the two heavy chain-light chain pairs are disulfide bonded to one another via the heavy chains in each pair. X-ray studies have suggested that the triggering of effector function by antibodies which have bound to antigen is mediated by the C_{H}2 and hinge regions, especially the hinge region (Radaev et al. (2001), J. Biol. Chem. 276, 16469-16477). Hinge, C_{H}2 and C_{H}3 regions from both heavy chains make up the so-called Fc region of the antibody.

A major effector function is antibody-dependent cellular cytotoxicity (“ADCC”). ADCC is mediated by a bifunctional binding activity of IgG1. Via its Fc domain, the antibody transiently tethers Fc-gamma receptor (“Fc-gamma-R”)-positive cytotoxic immune cells to antibody-decorated cells carrying the antigen bound by antibody. This leads to formation of a cytolytic synapse between cells, a targeted delivery of cytotoxic proteins, such as perforin and granzymes by the immune cell and, ultimately, induction of lysis of the cell carrying the antigen bound. The cell carrying the antigen bound may for example be an endogenous cell, such as a tumor cell or other cells involved in pathogenic conditions. The endogenous cells may also for example be a cell which has become infected with a pathogen, for example a virus. The cell carrying the antigen may also be a an exogenous cell or pathogen, for example a bacterium invading the human body. Important immune cells participating in ADCC are i.a. natural killer (NK) cells bearing the low affinity Fc-gamma-R IIIa (CD16). Effector cells involved in ADCC include neutrophils, monocytes, macrophages, dendritic cells and natural killer cells. NK cells are probably the key cells in ADCC. This notion is genetically supported by a correlation of reduced efficacy of rituximab (an anti-CD20 antibody) with a polymorphism in CD16 that reduces the receptor's affinity for antibody ligand (Cartron et al. (2002) Blood 99, 754-8). A similar correlation was identified for a polymorphism of CD32 (Weng & Levy (2003) J. Clin. Oncol. 21, 3940-7), suggesting that CD32-positive immune cells also contribute to ADCC.

In order to be marketed, any new candidate antibody drug must pass through rigorous testing. Roughly, this testing can be subdivided into preclinical and clinical phases: Whereas clinical testing—further subdivided into the generally known clinical phases I, II and III—is performed in human patients, preclinical testing is performed in animals. Generally, the aim of preclinical testing is to prove that the antibody drug candidate works at all and is safe, i.e. that a safety margin exists between the efficacious dose and the toxic dose, or the maximal tolerated dose (“MTD”). Specifically, the purpose of these animal studies is to make a risk assessment and to prove that the drug is not carcinogenic, mutagenic or teratogenic, as well as to understand the pharmacokinetics of the drug candidate. Only when it has been established that the drug candidate a) is not toxic to the test animal at therapeutic doses and b) shows signs of efficacy in the test animal (regardless of their magnitude), will this drug candidate be approved for clinical testing in humans.

In preclinical testing, the efficacy of human IgG1 therapies is frequently assessed in xenotransplant models employing mice as test animals. Mice have a short life cycle, reproduce frequently, are not an endangered species, may be relatively easily genetically manipulated and are easy and cheap to maintain. They are thus highly advantageous animals for preclinical testing.

One kind of animal model frequently used in preclinical testing is an immunodeficient nude mouse, for example an SCID mouse. Due to the extremely low levels of T cells in nude mice, human cell lines (for example tumor cell lines) expressing the corresponding antigen bound by the antibody drug candidate may be introduced into such mice without being rejected by the mouse immune system. In the event that the injected human cell line is a human tumor cell line, this cell line grows into measurable tumors in such mice. Although T cells are present in only very low amounts in nude mice, other effector cells such as NK cells and/or granulocytes remain despite the mouse's immunodeficiency. These effector cells are able to mediate ADCC, offering a useful preclinical readout for the efficacy of the antibody drug candidate.

Other types of mouse models used in preclinical testing are immunocompetent mouse models. In these models, syngeneic tumor cell lines transfected with the human target antigen are intravenously injected, become trapped in lung capillaries and subsequently grow to macroscopically visible lung tumor colonies.

As is known in the art, it is desirable that an antibody drug candidate intended for administration to humans does not trigger an immune response in the patient, i.e. it is desirable that this antibody is a human antibody or is at least derived from a human antibody. Human antibodies intended for therapy are known in the art, for example as described in WO 98/46645.

However, a drawback of performing preclinical testing of a human antibody, especially a human IgG1 antibody, in any kind of mouse model is that the Fc-gamma receptors of mouse immune effector cells do not properly recognize the Fc domain of a human antibody. As a result, the data obtained in preclinical testing is often not predictive of what would be expected when administering the same antibody to a human patient whose Fc-gamma receptors properly recognize the Fc domain of human antibody. The result is a falsification of the readout, for example the intensity of ADCC, obtained from the preclinical test-animal. This can have several consequences, each of them adverse to the development of the antibody drug candidate.

One possibility is that very little or no ADCC at all is measured in preclinical testing. Failing any indication of efficacy, it is unlikely that the antibody drug candidate would be admitted to the clinical phase of testing in humans, even though the drug candidate might actually have been efficacious in a human patient. In this case, a potentially promising antibody drug candidate may be needlessly abandoned.

Another possibility is that, due to an improper recognition of the Fc portion of the antibody drug candidate, an artificially high level of ADCC is measured. The danger here is that the antibody drug candidate—if also deemed safe—is carried further into the clinical phase of testing, where it would be established under conditions of proper molecular recognition in a human context that the antibody actually is insufficiently efficacious. In this case, precious money and time may be wasted pursuing an unworthy antibody drug candidate.

One known approach to solve this problem is to use an animal species which is genetically very close to humans for preclinical testing. Chimpanzees bear over 99% genetic identity to humans and are therefore the first choice for such preclinical testing. However, testing in chimpanzees is very expensive. In addition, leaving cost issues aside, chimpanzees are endangered creatures, so the number of animals which can be used in experimentation is very limited, thus reducing the statistical significance of any preclinical data obtained. The preclinical researcher must therefore resort to testing in other animal species more genetically distant from humans than the chimpanzee. Even for many non-chimpanzee primate species this phylogenetic distance from humans may often be so great as to render the resulting preclinical data obtained using a human antibody drug candidate inapplicable to a human setting.

It is a goal of the invention to provide a way of obtaining preclinical data for an antibody intended for human therapy in a reliable, flexible and cost-effective manner.

Accordingly, one aspect of the invention relates to a domain-grafted antibody which specifically binds a human cell-surface molecule. The domain-grafted antibody comprises an antibody heavy chain variable region (VH) of human origin; an antibody light chain variable region (VL) of human origin; a second antibody heavy chain constant region (C_{H}2) from a non-human species; and an antibody heavy chain hinge region from said non-human species. The antibody heavy and light chain variable regions of the domain-grafted antibody together define a binding site for said human cell-surface molecule.

A “domain-grafted antibody” is therefore an antibody comprising at a minimum VH, VL, C_{H}2 and hinge regions, the characteristics of which are set out in the preceding paragraph. Generally, it has been found that the accuracy of preclinical data obtained using accepted test animals (for example rodents) can be greatly increased by modifying the portion of a human antibody drug candidate which interacts with the Fc-gamma-R of the test animal's effector cells so as to be of the same origin as the particular animal species used for testing. An inventive domain-grafted antibody thus obtained for preclinical testing therefore has antigen binding regions (VH and VL) of human origin corresponding to those present in the actual antibody drug candidate, while at least the C_{H}2 and hinge regions of the heavy chain—those regions which most closely interact with the Fc-gamma-R of immune effector cells—are of test-animal origin. In this way, an antibody molecule is obtained which corresponds exactly or at least very closely in antigen binding ability to the actual antibody drug candidate to be approved for marketing. At the same time, the domain-grafted antibody remains compatible with the immune system of the preclinical test-animal. The safety and efficacy data thus obtained will more accurately represent that expected in the biologically relevant context of compatibility between antibody Fc portion and immune system. The compatibility between the domain-grafted antibody and Fc receptor thus obtained in a test animal is analogous to the compatibility between the Fc portion of the actual drug candidate and the human immune system when this candidate is later administered to a human patient. A schematic representation of such an inventive domain-grafted antibody is shown in FIG. 2, in which VH and VL (shaded) are of human origin, and C_{H}2 and hinge regions are from a (i.e. the same) non-human species. Interchain disulfide bonds are depicted by dotted lines and glycosylation sites are depicted by small hexagons.

A domain-grafted antibody as depicted in FIG. 2 has several distinct advantages.

First, the ability to adapt the effector-relevant portions of a human antibody to the animal in which preclinical testing is to take place circumvents the need to develop new test-animal species. For reasons outlined above, mice are most often used as at least one of the preclinical test species. The development of a mouse species which would be compatible with the human antibody for which regulatory approval is sought, for example a transgenic mouse species which expresses human Fc-gamma receptors on its immune effector cells, is generally protracted and costly, taking between 2-3 years. By comparison, production of a domain-grafted antibody according to the invention is quicker and cheaper, as the recombinant DNA technology required is well established and suitable mouse Fc domains are known in the art for each possible antibody isotype, an example being the Fc domain of OKT3, a well known murine antibody of the IgG2a format.

An additional advantage of the domain-grafted antibody of the invention is the degree of flexibility in acquiring the preclinical data required by regulatory authorities. Both in Europe and in the United States, the EMEA and FDA normally require preclinical data to be obtained in at least two different animal species. Since recombinant DNA technology makes it possible to attach any number of Fc portions from animals of different species to identical VH and VL regions of human origin, variously domain-grafted antibodies of the type depicted in FIG. 2 may be rapidly generated for a variety of different test-animal species.

This flexibility in antibody construction allows a higher statistical significance of the preclinical data obtained from a variety of different animal species. For example, preclinical safety data obtained from multiple animal species will have a higher probability of predicting safety in humans than data obtained from just one animal species. Similarly, preclinical data suggesting some degree of efficacy in multiple test-animal models will indicate efficaciousness in humans with a higher probability than preclinical data obtained from just one or two animal models.

As used herein, the term “specifically binds” or related expressions such as “specific binding”, “binding specifically”, “specific binder” etc. refer to the ability of the domain-grafted antibody to discriminate between an intended human cell-surface molecule and any number of other potential molecules different from said human cell-surface molecule to such an extent that, from a pool of a plurality of different antigens as potential binding partners, only said human cell-surface molecule is bound, or is significantly bound. Within the meaning of the invention, the human cell-surface molecule is “significantly” bound when, from among a pool of a plurality of equally accessible different molecules as potential binding partners, the intended human cell-surface molecule is bound at least 10-fold, preferably 50-fold, most preferably 100-fold or greater more frequently (in a kinetic sense) than any other molecule different than this human cell-surface molecule. Such kinetic measurements can be routinely performed on a Biacore apparatus or by Scatchard plot analysis.

The term “cell-surface molecule” as used herein denotes a molecule which is displayed on the surface of a cell. In most cases, this molecule will be located in or on the plasma membrane of the cell such that at least part of this molecule remains accessible from outside the cell in tertiary form. A non-limiting example of a cell-surface molecule which is located in the plasma membrane is a transmembrane protein comprising, in its tertiary conformation, regions of hydrophilicity and hydrophobicity. Here, at least one hydrophobic region allows the cell-surface molecule to be embedded, or inserted in the hydrophobic interior of the plasma membrane of the cell while the hydrophilic regions extend on one or both side(s) of the plasma membrane into the (hydrophilic) cytoplasm and/or extracellular space, respectively. Non-limiting examples of cell-surface molecules which are located on the plasma membrane are proteins which have been modified at a cysteine residue to bear a palmitoyl group, proteins modified at a C-terminal cysteine residue to bear a farnesyl group or proteins which have been modified at the C-terminus to bear a glycosyl phosphatidyl inositol (“GPI”) anchor. These groups allow covalent attachment of proteins to the outer surface of the plasma membrane, where they remain accessible for recognition by extracellular molecules such as antibodies.

As stated above, the “cell-surface molecule” specifically bound by the domain-grafted antibody of the invention is a human cell-surface molecule. This means that the cell-surface molecule has the same amino acid sequence and conformation that it has in vivo in humans. The fact that the cell-surface molecule is “human” does not, however, mean that this molecule must be present in living human beings. For example, and as explained in greater detail below, the human cell-surface molecule may be expressed in a non-human transgenic animal in the same form as this molecule is normally expressed in humans, and still be regarded as a “human cell-surface molecule” in the sense of the present invention.

The domain-grafted antibody of the invention comprises VH and VL regions “of human origin”. As used herein, this means that these parts of the domain-grafted antibody are human or humanized.

A “human” VH and/or VL region is a VH and/or VL region as it appears in either polypeptide or polynucleotide form in the human body. For example, a “human” VH may be a VH as comprised in an antibody displayed on the surface of a human B cell (regardless of whether or not this human B cell exists in vivo or in vitro), or may result from expression of the cDNA obtained by reverse transcribing the mRNA present in such a human B cell, for example by PCR. As another example, a “human” VH may alternatively be obtained by expression of germline antibody genes, or germline antibody genes having been imprinted to any degree by somatic hypermutation (Neuberger & Milstein (1995). Curr. Opin. Immunol. 7, 248-54).

A “humanized” VH and/or VL region is a VH and/or VL region in which at least one complementarity determining region (“CDR”) is from a non-human antibody or fragment thereof. Humanization approaches are described for example in U.S. Pat. No. 5,225,539 and EP 0 239 400 B1. As non-limiting examples, the term encompasses the case in which the VH and VL each comprise a single CDR region from another non-human animal, for example a rodent, as well as the case in which a or both variable region/s comprise at each of their respective first, second and third CDRs the corresponding CDRs from a non-human animal.

In the event that CDRs of a binding domain of an antibody have been replaced by their corresponding equivalents in a non-human antibody, one typically speaks of “CDR-grafting”, and this term is to be understood as being encompassed by the term “humanized”. The term “humanized” or grammatically related variants thereof also encompasses cases in which, in addition to replacement of one or more CDR regions within a VH and VL of the domain-grafted antibody, a further mutation (e.g. substitution) of at least one single amino acid residue within the FR has been effected such that the amino acid at that position is the same as the amino acid at the same position in the non-human animal from which the CDR regions used for replacement are taken. As is known in the art (see for example U.S. Pat. No. 5,859,205), such mutations are often made in the FR regions following CDR-grafting in order to restore the binding affinity of the humanized antibody for its antigen to the level of that observed for the non-human antibody used as a CDR-donor.

As used herein, the term “binding site” denotes the single site at the tip of each arm of the domain-grafted antibody formed when the VH and VL comprised therein are paired together. In this pairing, the hypervariable regions, i.e. the CDR regions, from each of the VH and VL regions are brought together in a tertiary structure which is complementary to the molecule, or antigen to be bound. FR regions within the VH and VL also play a crucial role in the tertiary positioning of the CDR regions, so that these FR regions may also be considered part of the “binding site” as used herein. However, it is generally the amino acids in the individual CDRs which make contact with the molecule, or antigen bound, for example with a cell-surface molecule. The amino acids of the molecule, or antigen, are therefore involved in interaction with the amino acids of the CDRs of the antibody molecule. The “binding site” of an antibody is also typically termed the “antibody combining site”. A “binding site” as used herein is described in more detail in sections 3-6-3-9 of “ImmunoBiology” Fifth Edition; Charles Janeway, Paul Travers, Mark Walport and Mark Shlochik; Published by Garland Publishing, 2001.

According to one embodiment of the invention, the domain-grafted antibody further comprises a third antibody heavy chain constant region (C_{H}3) from a non-human species. The resulting domain-grafted antibody according to this embodiment of the invention thus comprises an antibody heavy chain comprising, N→C: a VH, an antibody hinge region, a second antibody heavy chain constant region (C_{H}2) and a third antibody heavy chain constant region (C_{H}3); and an antibody light chain comprising a VL. The domain-grafted antibody according to this embodiment of the invention therefore comprises a paired VH and VL of human origin and heavy chain hinge, C_{H}2 and C_{H}3 regions originating from the same non-human species. The further incorporation of the C_{H}3 region into the domain-grafted antibody according to this embodiment of the invention has the advantage of further fostering recognition of the Fc region of the domain-grafted antibody by Fc receptors which may bind to the C_{H}3 region in addition to the C_{H}2 and hinge regions of the heavy chain. Since according to this embodiment the hinge, C_{H}2 and C_{H}3 regions of the heavy chain of the domain-grafted antibody originate from the same non-human species, i.e. from the non-human animal species used for preclinical testing, a better recognition of the domain-grafted antibody by Fc-gamma-R of the test animal species is achieved. This translates into more accurate preclinical test data with a higher transferability to the scenario in which an antibody intended for market approval and comprising a human Fc region would be administered to a human. A schematic representation of the domain-grafted antibody according to this embodiment is shown in FIG. 3, in which VH and VL (shaded) are of human origin, and C_{H}2, C_{H}3 and hinge regions are from a non-human species. Interchain disulfide bonds are depicted by dotted lines and glycosylation sites are depicted by small hexagons.

According to a further embodiment of the invention, the domain-grafted antibody further comprises a first antibody heavy chain constant region (C_{H}1) from said non-human species and an antibody light chain constant region (C_{L}) from said non-human species. A domain-grafted antibody according to this embodiment of the invention may thus minimally comprise an antibody heavy chain comprising, N→C: a VH, a first antibody heavy chain constant region (C_{H}1), an antibody hinge region, a second antibody heavy chain constant region (C_{H}2) and a third antibody heavy chain constant region (C_{H}3); and an antibody light chain comprising, N→C: a VL and an antibody light chain constant region (C_{L}). The domain-grafted antibody according to this embodiment of the invention may therefore be a full immunoglobulin molecule in which VH and VL are of human origin and all other regions are from the same non-human species, i.e. the species of non-human animal used for preclinical testing purposes. A schematic representation of the especially preferred domain-grafted antibody which does not omit any constant regions is shown in FIG. 1, in which VH and VL (shaded) are of human origin, and all other regions are from a non-human species. Interchain disulfide bonds are depicted by dotted lines and glycosylation sites are depicted by small hexagons.

Alternatively, the domain-grafted antibody according to this embodiment of the invention may omit the C_{H}3 region but comprise the C_{H}1 and C_{L} regions. In effect, such a domain-grafted antibody amounts to a full antibody with truncated C_{H}3 region, all variable regions of which are of human origin, and all other regions of which are from the same non-human species, i.e. the species of non-human animal used for preclinical testing purposes. However, regardless whether or not the C_{H}3 region is present, the C_{H}1 and C_{L} regions of antibodies pair together, so when incorporating a C_{H}1 region into a domain-grafted antibody according to this embodiment of the invention, a C_{L} region should also be incorporated. Conversely, when incorporating a C_{L} region into a domain-grafted antibody according to this embodiment of the invention, a C_{H}1 region should also be incorporated.

A schematic representation of a domain-grafted antibody with CHI and C_{L} regions but not C_{H}3 regions is shown in FIG. 4, in which VH and VL (shaded) are of human origin, and C_{H}2, C_{H}1, C_{L} and hinge regions are from the same non-human species i.e. the species of non-human animal used for preclinical testing purposes. Interchain disulfide bonds are depicted by dotted lines and glycosylation sites are depicted by small hexagons.

Antibody light chains are known in the art to exist as kappa light chains and lambda light chains. In the context of the present invention, it is preferred that the antibody light chain components of the domain-grafted antibody are of the kappa type.

In this embodiment of the invention, it is especially preferred to additionally incorporate a C_{H}3 region as well as the CHI and C_{L} regions; i.e. a domain-grafted antibody is especially preferred which is a full immunoglobulin in which the VH and VL are of human origin and all other regions (C_{H}1, C_{L}, C_{H}2, C_{H}3 and hinge) are from the same non-human species, i.e. the species of non-human animal used for preclinical testing purposes (shown schematically in FIG. 1). There are several particular advantages in forming the domain-grafted antibody in this way, i.e. as a full antibody. First, as already explained above under the previous embodiment, the incorporation of a test animal-species C_{H}3 region allows recognition by a Fc-gamma-R in the test animal which may be more congruent in effect to that observed when administering antibodies with human Fc regions to humans. However, a full, domain-grafted antibody, i.e. a domain-grafted antibody in which no region has been omitted as compared to an Ig antibody intended for market approval, will also have a very similar molecular weight as the corresponding antibody intended for market approval. Molecular weight of antibodies is generally related to the rate at which such antibodies are excreted from the bodies of humans and test animals alike; a larger antibody will generally be cleared from the body more slowly than a smaller one. This means that the preclinical data obtained using a full, domain-grafted antibody according to this embodiment of the invention potentially enables highly comparable information not only with regard to safety and efficacy as explained hereinabove, but also regarding pharmacokinetics.

Especially preferred within this embodiment is a domain-grafted antibody with a heavy chain having an amino acid sequence as set out in SEQ ID NO. 2 and with a light chain having an amino acid sequence as set out in SEQ ID NO. 4. SEQ ID NO.2 and SEQ ID NO. 4 may be encoded by DNA sequences as set out in SEQ ID NO.1 and SEQ ID NO. 3, respectively, although one of ordinary skill in the art will understand that there exist many potential DNA sequences which may encode the respective amino acid sequences of SEQ ID NO.2 and SEQ ID NO. 4, due to the degeneracy of the genetic code. As such any polynucleotide sequence, for example any DNA sequence encoding an amino acid sequence as set out SEQ ID NO.2 and SEQ ID NO. 4, respectively, is to be considered as being encompassed within this embodiment of the invention.

When associated with one another, the heavy and light antibody chains given by SEQ ID NO.2 and SEQ ID NO. 4, respectively, form an antibody specific for the human cell surface molecule EpCAM, a molecule expressed on a wide range of human epithelial cancers, and which becomes more accessible in a cancerous state than in a non-cancerous state. This domain-grafted antibody is a full antibody, meaning that its heavy chain comprises VH, C_{H}1, hinge, C_{H}2 and C_{H}3 regions, while its light chain comprises VL and C_{L} regions. The VH and VL regions are human (as defined above), and are comprised in the known anti-EpCAM antibody Anti-EpCAM (see WO 98/46645), while all constant regions and the hinge region are of murine origin, specifically of the IgG2a isotype in the known anti-CD3 antibody OKT3 (see for example U.S. Pat. No. 5,885,573). In the following, the domain grafted antibody given by the combination of polypeptides with amino acids as set out in SEQ ID NOs. 2 and 4 is referred to as domain-grafted Anti-EpCAM, or dgAnti-EpCAM.

According to a further embodiment of the invention, the VH and VL of human origin may be human or humanized.

Within this embodiment, and as described briefly above, the term “human” is to be understood as denoting a part of an antibody as it appears in either polypeptide or polynucleotide form in the human body. For example, a “human” VH may be a VH as comprised in an antibody displayed on the surface of a human B cell (regardless of whether or not this human B cell exists in vivo or in vitro), or may result from expression of the cDNA obtained by reverse transcribing the mRNA present in such a human B cell, for example, as is known in the art using phage display technology based on a library or on libraries of VH and VL genes obtained via PCR from human B cells, for example human IgD cells (Raum, T., et al. (2001) Cancer Immunol. Immunother. 50, 141-50). A “human” VH may alternatively be obtained by expression germline antibody genes or germline antibody genes which have been imprinted to any degree by somatic hypermutation. The degree to which the a human VH or/and VL correspond in sequence to the human germline sequences encoding these regions increases the closer the human cells used as a source of these genes are in hematopoietic development to pluripotent hematopoietic stem cells. Conversely, the further the human cells used for obtaining human VH and/or VL regions are in hematopoietic development from pluripotent hematopoietic stem cells, the more one may expect the VH and VL sequences obtained to differ from the corresponding human germline sequences. This is because as hematopoiesis progresses and the individual cells in the lymphoid line mature, they are subject to increasing degrees of somatic hypermutation. Such cells are said to bear the “imprint of somatic hypermutation”, a process by which variation is introduced over time into rearranged antibody variable regions subject to negative and positive selection in order to yield improved binding to antigen. This process is known in the art and is described in detail in section 4-9 of “ImmunoBiology” Fifth Edition; Charles Janeway, Paul Travers, Mark Walport and Mark Shlochik; Published by Garland Publishing, 2001 as well as in Neuberger & Milstein (1995) Curr. Opin. Immunol. 7, 248-54.

Within this embodiment of the invention, the terms “humanized,” “humanization” or grammatically related variants thereof are used interchangeably to refer to a VH or VL region in which at least one complementarity determining region (“CDR”) is from a non-human antibody or fragment thereof. Methods are well known in the art for the humanization of antibodies i.a. by CDR grafting (see for example U.S. Pat. No. 5,225,539). A CDR-grafted humanized antibody is one which comprises CDR regions from an antibody produced in a non-human animal, often a rodent such as a mouse or a corresponding hybridoma cell. These CDR regions are set within a FR which originates from a human antibody or a human antibody-producing cell. The resulting variable region demonstrates the same ability to bind to an antigen as the non-human antibody from which the CDRs were obtained. At the same time, this variable region will be much less immunogenic in humans than the corresponding variable region of the non-human antibody, due to the preponderance of human amino acid sequences contained in the human FR regions. As is known in the art, it is sometimes necessary to mutate certain amino acids in the otherwise human FR regions to be the same as the amino acids at that/those position(s) in the non-human antibody from which the CDRs were taken. Such mutation may be necessary to ensure proper folding of the non-human CDRs in the now humanized antibody and, thus, proper antigen recognition (as for example described in U.S. Pat. No. 6,407,213 and U.S. Pat. No. 5,859,205). CDR-grafted VH and VL regions both with and without additional mutations in the otherwise human FR are to be understood as being “humanized” within the meaning of this embodiment of the invention. It is also envisioned in this embodiment of the invention that a VH and/or VL in which only one non-human CDR has been substituted into a human FR, is/are to be considered “humanized,” regardless of whether or not additional mutations within the FR are present.

According to a preferred embodiment of the antibody of the invention the antibody heavy and light chain variable regions of human origin are independently human or humanized.

According to a further embodiment of the invention, the non-human species may be a rodent species, a non-human primate species, rabbit, beagle dog, pig, mini-pig, goat or sheep. The C_{H}2, hinge region and, as the case may be, C_{H}3, C_{H}1 and/or C_{L} regions may therefore be from one of these species. Since the inventive domain-grafted antibody is intended for administration to a particular test animal, it makes most sense that the origin of all non-variable regions comprised in the domain-grafted antibody be of the same non-human species of animal. Particularly preferred rodent species are mouse, rat, guinea pig, hamster or gerbil. When the rodent species is mouse, it is particularly preferred that all constant regions comprised in the domain-grafted antibody, i.e. C_{H}2, hinge region and, as the case may be, C_{H}3, C_{H}1 and C_{L} regions, be of the gamma isotype. The resulting preferred domain-grafted antibody is therefore an IgG. In mouse, the gamma subclass is subdivided into gamma 1, gamma 2a, gamma 2b and gamma 3. An especially preferred domain-grafted antibody comprises constant regions belonging to the subclass gamma 2a. The mouse subclass gamma 2a (i.e. IgG2a) corresponds most closely to the human subclass gamma 1 (i.e. IgG1), the type of human IgG about which the most is known. Most known antibody pharmaceuticals are of the IgG1 type. A domain-grafted antibody of the mouse type IgG2a therefore has the advantage of high preclinical comparability to a human IgG1 therapeutic for which market approval is sought.

According to a further embodiment of the invention, the non-human primate species may be a chimpanzee, cynomolgous monkey, rhesus monkey, baboon or marmoset. For reasons set out above, it is often advantageous to avoid using a chimpanzee as a test animal species for preclinical testing, however there are certain cases in which use of a chimpanzee may be indicated. It is especially advantageous that all constant regions comprised in the domain-grafted antibody, i.e. C_{H}2, hinge region and, as the case may be, C_{H}3, C_{H}1 and C_{L} regions come from cynomolgous monkey or rhesus monkey. Due to their relative phylogenetic proximity to human beings, these species are accepted animals for use in preclinical testing.

According to a further embodiment of the invention the human cell-surface molecule is exclusively or overexpressed in a pathological state or is more readily accessible for recognition by specific antibodies in a pathological state than in a non-pathological state. As used in this embodiment of the invention, a “pathological state” is to be understood as a state of dysfunction or disease relative to a healthy state. The state of disease or dysfunction may be engendered by either internal or external factors. A non-limiting example of an internal factor may be excess cell proliferation and tissue growth as in cancer. A non-limiting example of an external factor may be an infection brought about by an organism foreign to the diseased system, for example a bacterium, virus or parasite. Often, a human cell-surface molecule which is normally expressed in a healthy individual to a certain degree on cells of a specific type is expressed to a much higher degree on these cells when the individual is in a pathological state. Assuming that the cells expressing this human cell-surface molecule contribute to the existence and development of the pathological state, this high expression may be therapeutically exploited by an antibody medication targeting such cells for destruction. In other types of pathological states, a human cell-surface molecule which normally remains inaccessible, or whose accessibility is limited, becomes (more) accessible in a pathological state for binding of specific antibodies. An example of such human cell-surface molecules are molecules involved in cell-cell adhesion, which in a healthy individual remain in tight association with one another, but which, in cancer, become accessible due to the disintegration of the cancerous tissue structure. As explained above, this (increased) accessibility may be therapeutically exploited by an antibody medication targeting such cells for destruction.

In a preferred embodiment of the invention, the pathological state is a proliferative disease, preferably a tumorous disease. Within this embodiment of the invention, a “tumorous disease” is to be understood as a disease involving increased cell proliferation and abnormal growth of tissue; this growth may be of a benign or a malignant nature. It is especially preferred that this growth is of a malignant nature, i.e. that the tumorous disease be a cancerous disease. Such a disease is characterized not only by abnormal tissue growth but also by invasion of this abnormal growth into surrounding or distant healthy tissue (the latter via metastasis), accompanied by destruction of this healthy tissue.

According to a further preferred embodiment of the invention, the human cell-surface molecule in a cancerous disease is human EpCAM or human CD25.

According to a further embodiment of the invention the pathological state is a pathogen-related disease. As explained above, a “pathogen-related disease” is to be understood within this embodiment of the invention as a disease caused or aggravated by an organism foreign to the diseased individual. For example, such a foreign organism may be a virus, a bacterium or a parasite, the latter of which may be unicellular (as for example in plasmodium-type infections) or multicellular (as for example in nematode infections). A preferred pathogen-related disease is a viral disease or a retroviral disease. Preferred viral diseases are for example caused by herpes simplex virus (HSV), human papilloma virus (HPV), cytomegalovirus. (CMV) or Epstein-Barr Virus (EBV). A preferred retroviral disease is caused by human immunodeficiency virus (HIV), human T cell leukaemia virus 1 (HTLV-1) or human T cell leukaemia virus 2 (HTLV-2).

According to an also preferred embodiment of the invention, the pathological state is an inflammatory disease. Within this embodiment of the invention, an “inflammatory disease” is to be understood as a condition characterized or caused by swelling, redness, heat and/or pain produced in an area of the body as a result of irritation, injury or infection. Within this embodiment of the invention, the human cell-surface molecule may be a human membrane-bound IgE molecule. Within this embodiment of the invention relating to inflammatory disease, preferred classes of human cell-surface molecules are those belonging to chemokine receptors, cytokine receptors or c-type lectin receptors. Especially preferred, the cytokine receptor is the human granulocyte-macrophage colony stimulating factor (GM-CSF) receptor, or human CCR5. An especially preferred c-type lectin receptor is human NKG2D.

According to a further embodiment of the invention, the pathological state is an autoimmune disease. Within this embodiment of the invention, an “autoimmune disease” is to be understood as a condition in which the body's immune system mistakenly attacks and destroys body tissue that it believes to be foreign. Within this embodiment of the invention, the human cell-surface molecule may be human ICOS, human CTLA4, human PD1, human CCR8 or human CCR3.

A further aspect of the invention is a pharmaceutical composition comprising a domain-grafted antibody as described hereinabove. In accordance with this aspect of the invention, the term “pharmaceutical composition” relates to a composition for administration to a mammalian patient, preferably a human patient. In a preferred embodiment, the pharmaceutical composition comprises a composition for parenteral injection or infusion. Such parenteral injection or infusion may take advantage of a resorption process in the form of e.g. an intracutaneous, a subcutaneous, an intramuscular and/or an intraperitoneal injection or infusion. Alternatively, such parenteral injection or infusion may circumvent resorption processes and be in the form of e.g. an intracardial, an intraarterial, an intraveneous, an intralumbal and/or an intrathecal injection or infusion. In another preferred embodiment, the pharmaceutical composition comprises a composition for administration via the skin. One example of administration via the skin is an epicutaneous administration, in which the pharmaceutical composition is applied as e.g. a solution, a suspension, an emulsion, a foam, an unguent, an ointment, a paste and/or a patch to the skin. Alternatively, administration of the pharmaceutical composition may be effected via one or more mucous membranes. For example, administration may be buccal, lingual or sublingual, i.e. via the mucous membrane(s) of the mouth and/or tongue, and may be applied as e.g. a tablet, a lozenge, a sugar coated pill (i.e. dragée) and/or as solution for gargling.

Alternatively, administration may be enteral, i.e. via the mucous membrane(s) of the stomach and/or intestinal tract, and may be applied as e.g. a tablet, a sugar coated pill (i.e. dragée), a capsule, a solution, a suspension and/or an emulsion. Alternatively, administration may be rectal, and may be applied as e.g. a suppository, a rectal capsule and/or an ointment or unguent. Alternatively, administration may be intranasal, and may be applied as e.g. drops, an ointment or unguent and/or a spray. Alternatively, administration may be pulmonary, i.e. via the mucous membrane(s) of the bronchi and/or the alveolae, and may be applied as e.g. an aerosol and/or an inhalate. Alternatively, administration may be conjunctival, and may be applied as e.g. eye drops, an eye ointment and/or an eye rinse. Alternatively, administration may be effected via the mucous membrane(s) of the urogenital tract, e.g. may be intravaginal or intraurethal, and may be applied as e.g. a suppository, an ointment and/or a stylus. It should be understood that the above administration alternatives are not mutually exclusive, and that a combination of any number of them may constitute an effective therapeutic regimen.

The pharmaceutical composition of the present invention may further comprise a pharmaceutically acceptable carrier, or excipient. Examples of suitable pharmaceutical carriers are well known in the art and include phosphate buffered saline solutions, water, emulsions, such as oil/water emulsions, various types of wetting agents, and sterile solutions. Compositions comprising such carriers can be formulated by well known conventional methods. These pharmaceutical compositions can be administered to the subject at a suitable dose. The dosage regimen will be determined by the attending physician and clinical factors. As is well known in the medical arts, dosages for any one patient depend upon many factors, including the patient's size, body surface area, age, the particular compound to be administered, sex, time and route of administration, general health, and other drugs being administered concurrently. Preparations for e.g. parenteral administration include sterile aqueous or non-aqueous solutions, suspensions, emulsions and liposomes. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Vehicles suitable for general parenteral administration include sodium chloride solution, Ringer's dextrose, dextrose and sodium chloride, lactated Ringer's, or fixed oils. Vehicles suitable for intravenous or intraarterial administration include fluid and nutrient replenishers, electrolyte replenishers (such as those based on Ringer's dextrose), and the like. Preservatives and other additives may also be present such as, for example, antimicrobials, anti-oxidants, chelating agents, inert gases and the like. In addition, the pharmaceutical composition of the present invention might comprise proteinaceous carriers, like, e.g., serum albumin or immunoglobulin, preferably of human origin. It is envisaged that the pharmaceutical composition of the invention might comprise, in addition to the domain-grafted antibody (as described in this invention), further biologically active agents, depending on the intended use of the pharmaceutical composition. Such agents might be drugs acting on the gastro-intestinal system, drugs acting as cytostatica, drugs preventing hyperurikemia, drugs inhibiting immunoreactions (e.g. corticosteroids), drugs modulating the inflammatory response, drugs acting on the circulatory system and/or agents such as cytokines known in the art.

A further aspect of the invention relates to an expression vector encoding at least the heavy or light chain, or both, of the domain grafted antibody of the invention. Such an expression comprises:
a first coding sequence encoding: a) a heavy chain variable region of human origin, (b) a second antibody heavy chain constant region (CH2) from a non-human species and (c) an antibody heavy chain hinge region from said non-human species, and optionally, (d) a third antibody heavy chain constant region (CH3) from said non-human species and/or (e) a first antibody heavy chain constant region (CH1) from said non-human species and/or;
a second coding sequence encoding: a desired antibody light chain variable region (VL) of human origin and, optionally, an antibody light chain constant region (CL) from said non-human species;
   said antibody heavy and light chain variable regions together defining a binding site for a human cell-surface molecule.

The term “coding sequence” within this embodiment of the invention is to be understood as meaning a polynucleotide sequence which, when translated into a corresponding amino acid sequence, results in the combination of variable and constant regions comprised in the domain-grafted antibody. A “coding sequence” according to this aspect of the invention may be in the form of DNA or RNA. The DNA may contain or exclude introns, with cDNA (excluding introns) being an especially preferred form of the encoding sequence.

Specifically, the first coding sequence comprises, at a minimum, polynucleotides encoding a VH of human origin, a C_{H}2 region from a non-human species and an antibody hinge region from the same non-human species. Optionally, this first coding sequence may additionally comprise polynucleotides encoding one or both of C_{H}3 and C_{H}1 regions. The first coding sequence thus comprises polynucleotides encoding any antibody regions present in the domain-grafted antibody of the invention which are normally present as part of the antibody heavy chain of an IgG.

A second coding sequence comprises, at a minimum, a polynucleotide encoding a VL of human origin. Optionally, this second coding sequence may additionally comprise polynucleotides encoding a C_{L} region from the same non-human species as the polynucleotides encoding any antibody constant regions in the first coding species. The second coding sequence thus comprises polynucleotides encoding any antibody regions present in the domain-grafted antibody of the invention which are normally present as part of the antibody light chain of an IgG.

As explained above in the context of the domain-grafted antibody itself, when including a polynucleotide encoding an antibody C_{L} region into the second coding sequence, it is necessary to also include a polynucleotide encoding the C_{H}1 sequence into the first coding sequence, and vice versa. The resulting domain-grafted antibody will therefore comprise C_{H}1 and C_{L} regions.

Generally, the first coding sequence will encode all heavy chain-derived regions of the domain-grafted antibody while the second coding sequence will encode all light chain-derived regions of the domain-grafted antibody. First and second coding sequences are then incorporated into at least one expression vector. Separate incorporation of first and second coding sequences into two expression vectors will however generally be most advantageous, as doing so allows separate control over the expression of heavy chain- and light chain-derived components of the resulting domain-grafted antibody.

Within this aspect of the invention, the expression vector may be, for example, a phage, plasmid, viral or retroviral vector. Retroviral vectors may be replication competent or replication defective. In the latter case, viral propagation generally will occur only in complementing host/cells.

The expression vector of the invention may contain selectable markers for propagation in a host. Generally, a plasmid vector is introduced in a precipitate such as a calcium phosphate precipitate or rubidium chloride precipitate, or in a complex with a charged lipid or in carbon-based clusters, such as fullerenes. Should the vector be a virus, it may be packaged in vitro using an appropriate packaging cell line prior to application to host cells.

In one embodiment, the first and second coding sequences are operatively linked to expression control sequences allowing expression in prokaryotic or eukaryotic cells.

As explained above, expression of said first and second coding sequences comprises the possibility of transcription of these sequences, preferably into a translatable mRNA. Regulatory elements ensuring expression in eukaryotic cells, preferably mammalian cells, are well known to those skilled in the art. They usually comprise regulatory sequences ensuring initiation of transcription and optionally poly-A signals ensuring termination of transcription and stabilization of the transcript. Additional regulatory elements may include transcriptional as well as translational enhancers. Possible regulatory elements permitting expression in prokaryotic host cells comprise, e.g., the lac, trp or tac promoter in E. coli, and examples for regulatory elements permitting expression in eukaryotic host cells are the AOX1 or GAL1 promoter in yeast or the CMV-, SV40-, RSV-promoter (Rous sarcoma virus), CMV-enhancer, SV40-enhancer or a globin intron in mammalian and other animal cells. Besides elements which are responsible for the initiation of transcription, such regulatory elements may also comprise transcription termination signals, such as the SV40-poly-A site or the tk-poly-A site, downstream of the polynucleotide. In this context, suitable expression vectors are known in the art such as Okayama-Berg cDNA expression vector pcDV1 (Pharmacia), pCDM8, pRc/CMV, pcDNA1, pcDNA3 (Invitrogen), pSPORT1 (GIBCO BRL). Methods which are well known to those skilled in the art can be used to construct recombinant viral vectors; see, for example, the techniques described in Sambrook, Molecular Cloning A Laboratory Manual, Cold Spring Harbor Laboratory (1989) N.Y. and Ausubel, Current Protocols in Molecular Biology, Green Publishing Associates and Wiley Interscience, N.Y. (1994).

According to a further embodiment, the first and second coding sequences are present on the same or separate vectors. These same or separate vectors may be incorporated into a host cell, for example a prokaryotic host cell (such as an E. coli cell) or, preferably, a eukaryotic host cell (such as a mammalian, yeast or insect cell). It is especially preferred that expression be carried out in a mammalian cell, for example a CHO cell. This host cell may be cultivated so as to secrete the domain-grafted antibody, such as dgAnti-EpCAM, preferably under serum-free conditions. Following cultivation, the secreted domain-grafted antibody may be isolated and formulated into a pharmaceutical composition suitable for administration to a patient.

According to an especially preferred embodiment of this aspect of the invention, the first coding sequence comprises a nucleotide sequence as set out in SEQ ID NO: 1 and the second coding sequence comprises a nucleotide sequence as set out in SEQ ID NO: 3. Here it is especially preferred that the first coding sequence comprising SEQ ID NO: 1 is present on a first vector, while the second coding sequence comprising SEQ ID NO: 3 is present on a separate, second vector. These first and second vectors are preferably expression vectors suitable for expression in a mammalian host cell system, such as a CHO host cell system, preferably under serum-free conditions.

An alternative embodiment of the invention relates to a host cell comprising the expression vector of the invention. Moreover, the invention provides a method of producing a domain-grafted antibody of the invention. Said method comprises the step of culturing the host cell of the invention under conditions suitable for growth of said host cell. It is preferred that this host cell is a mammalian cell, more preferably a CHO cell. It is preferred for this method that the step of culturing is performed in serum-free medium. It is also preferred that the further comprises the step of isolating said domain-grafted antibody. More preferably, the method further comprises the purification of said domain-grafted antibody. According to a preferred embodiment of the method said domain-grafted antibody is formulated into a pharmaceutical composition in an additional step.

In a further alternative embodiment the invention relates to a pharmaceutical composition comprising the domain-grafted antibody of the invention or producible according to the method of the invention.

A further aspect of the invention relates to a method of measuring the in vivo activity of a pharmaceutical composition or a domain-grafted antibody as described hereinabove or obtainable as described hereinabove, said method comprising administering the composition or said domain-grafted antibody to a non-human animal expressing a human cell-surface molecule and measuring the in vivo activity of said composition or said domain-grafted antibody, wherein at least the second antibody heavy chain constant region (C_{H}2) and the antibody heavy chain hinge region of said domain-grafted antibody are from the same species of non-human animal as the non-human animal to which said domain-grafted antibody or composition is administered.

This aspect of the invention is especially advantageous in obtaining preclinical test data on the domain-grafted antibody which will be reflective of the safety and efficacy to be expected of the antibody intended for market approval when the latter is administered to human patients. The reasons for the high degree of applicability of these preclinical data to the human case are set out hereinabove.

According to one embodiment of this aspect of the invention, the non-human animal is advantageously a transgenic non-human animal. Within this embodiment “transgenic” means that this non-human animal has been genetically modified to express on at least one subpopulation of cells the human cell-surface molecule specifically bound by the antibody drug candidate and hence the domain-grafted antibody of the invention. While sometimes costly and time-consuming to produce (see above), a transgenic animal has the advantage that it may be used in preclinical testing without prior injection of a foreign cell line expressing said human cell-surface molecule. Since such transgenic animals have been modified only with regard to their ability to express the human cell-surface molecule specifically bound by the domain-grafted antibody of the invention, the immune system of such transgenic animals normally remains fully competent. This means that the transgenic animal has full populations of T cells, B cells and NK cells as well as other effector cells involved in antibody-mediated activities of the immune system. In conclusion, such transgenic animals, while sometimes somewhat expensive to produce, will generally produce preclinical safety and efficacy data which will be predictive of the corresponding antibody drug candidate intended for market approval when this is administered to a human patient.

According to a further, especially preferred embodiment of this aspect of the invention, the non-human animal is advantageously an immunocompetent non-human animal which is not transgenic. Like a transgenic non-human animal, the immunocompetent non-human animal has full populations of T cells, B cells and NK cells as well as other effector cells involved in antibody-mediated activities of the immune system. Unlike a transgenic non-human animal, however, an immunocompetent non-human animal does not endogeneously express the human cell-surface molecule bound by the domain-grafted antibody of the invention. In order for preclinical testing to take place, it is therefore first necessary to introduce this human cell-surface molecule into the immunocompetent non-human animal. Most frequently, this is accomplished by injecting at least one kind of cells or cell line expressing the human cell-surface molecule into said immunocompetent non-human animal. Preclinical testing measurements are then taken rapidly, before the test animal rejects the injected cells as foreign cells, something which often happens since the animal is immunocompetent.

In one embodiment of this aspect of the invention, the non-human animal is of rodent species, non-human primate species, a rabbit, a beagle dog, a pig, a mini-pig, a goat or a sheep. When the non-human animal is of rodent species, it is especially preferred that the animal of rodent species is a mouse, a rat, a guinea pig, a hamster or a gerbil. When the animal of rodent species is a mouse, as it will often be, this mouse may be a transgenic mouse (as described hereinabove), an immunocompetent mouse (as described hereinabove) or a nude mouse, for example a severe combined immunodeficient (“SCID”) mouse. Such mice are common models for preclinical testing purposes (Schultz et al. (1995) J. Immunol. 154, 180-91) and have severely compromised immune systems in which the levels of B cells and T cells have been completely or substantially reduced while NK cells and other effector cells remain present. As with transgenic mice, SCID-mice must be injected with at least one cell population expressing the human cell-surface molecule to which the domain-grafted antibody of the invention specifically binds. However, unlike with transgenic mice, the virtual absence of a functioning immune system in SCID-mice prevent such injected cells from being rejected as foreign. This extends the time within which meaningful preclinical test data may be collected. SCID-rats are also known in the prior art, and may be employed as a non-human rodent within this embodiment of the invention (Dekel et al. (1997) Transplant Proc. 29, 2255-6).

According to a further embodiment of this aspect of the invention, the animal of non-human primate species is a chimpanzee, a cynomolgous monkey, a rhesus monkey, a baboon or a marmoset, a cynomolgous monkey being especially preferred.

According to a further embodiment of the present method, the in vivo activity measured is in vivo cytotoxicity. For example the in vivo cytotoxicity may take the form of ADCC (see above for explanation) mediated by the domain-grafted antibody of the invention.

A further aspect of the invention relates to the use of a domain-grafted antibody as described hereinabove, or of a composition as described hereinabove for evaluating the functional in vivo biological activity of an antibody of human origin, wherein the domain-grafted antibody and the antibody of human origin (a) have identical VH and VL regions and (b) bind to the same human cell-surface molecule. As used in this aspect of the invention, an “antibody of human origin” is to be understood as an antibody drug candidate intended for market approval for administration to human patients, and which therefore contains sequences of human origin, especially—and in contrast to a domain-grafted antibody—in its constant regions (The term “of human origin” has been explained above in the context of VH and VL regions. The definitions of “of human origin” hereinabove apply as well to the antibody drug candidate intended for market approval as described within this aspect of the invention.) The “antibody of human origin” is thus the antibody which is to benefit from the preclinical testing in which the domain-grafted antibody of the invention is employed. In its broadest sense, the domain-grafted antibody is to be used as a surrogate antibody in preclinical animal testing, the results of which are highly predictive of the characteristics of the corresponding non-surrogate antibody (the “antibody of human origin”) when this non-surrogate antibody is administered to a human patient.

According to a further embodiment of the present use, the in vivo biological activity measured is in vivo cytotoxicity.

The invention will now be illustrated by the following figures and non-limiting examples.

### BRIEF FIGURE DESCRIPTION

FIG. 1 Schematic representation of an IgG molecule (comprising VH, C_{H}1, hinge, C_{H}2, C_{H}3, VL and C_{L} regions)

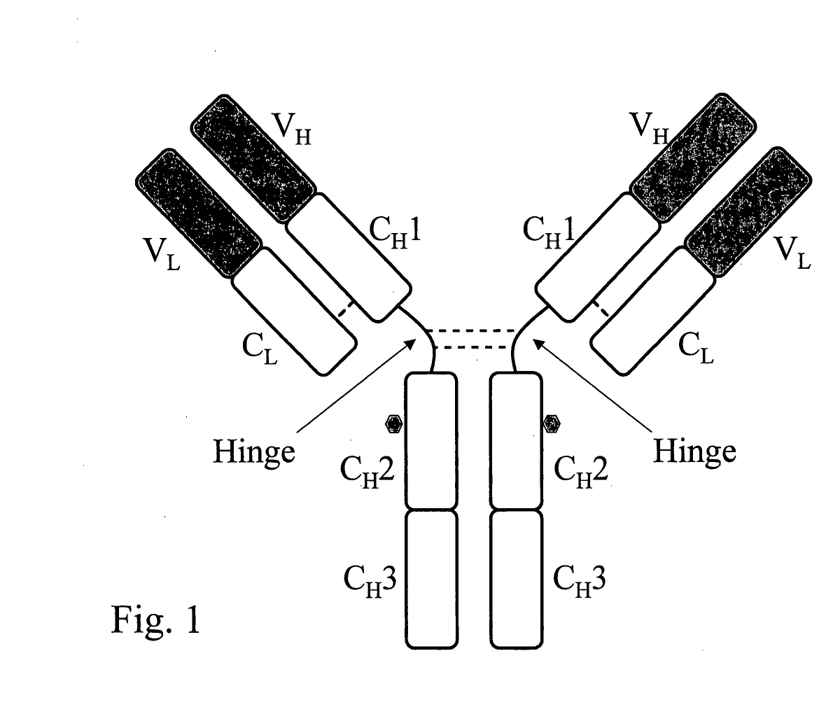

FIG. 2 Schematic representation of a domain-grafted antibody of the invention (comprising VH, hinge, C_{H}2 and VL regions)

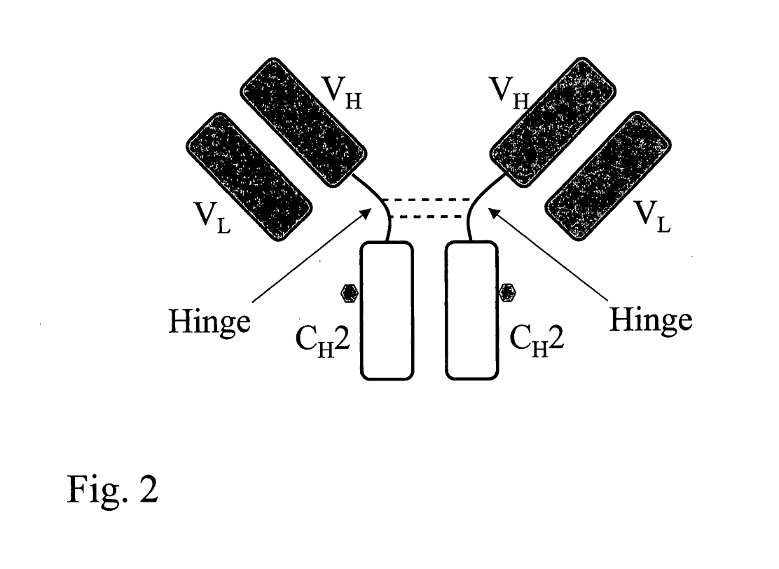

FIG. 3 Schematic representation of a domain-grafted antibody according to another embodiment of the invention (comprising VH, hinge, C_{H}2, C_{H}3 and VL regions)

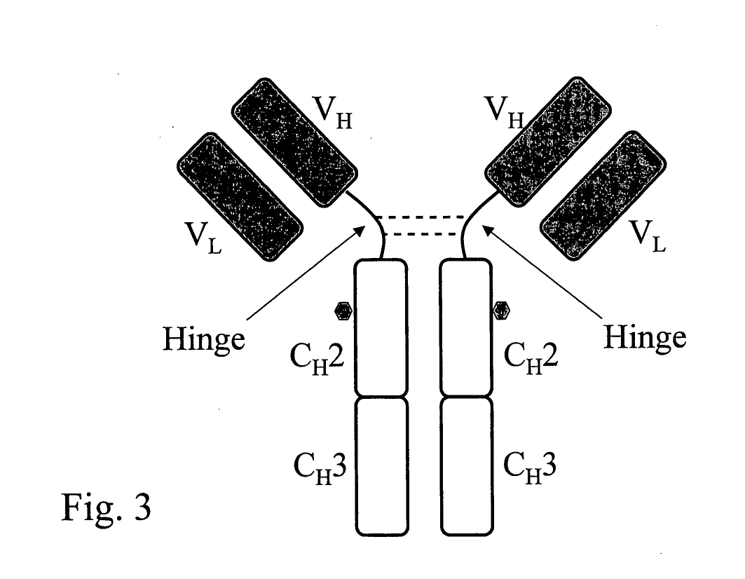

FIG. 4 Schematic representation of a domain-grafted antibody according to an embodiment of the invention (comprising VH, C_{H}1, hinge, C_{H}2, VL and C_{L} regions)

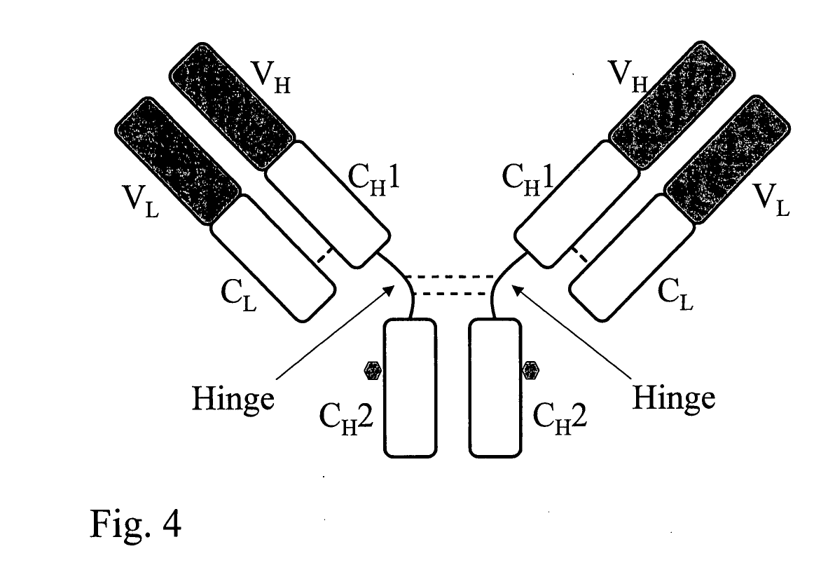

FIG. 5 Displacement of fluorescently labelled domain-grafted Anti-EpCAM (“dgAnti-EpCAM”) from human EpCAM-expressing Kato-III cells by non-labeled Anti-EpCAM, showing that dgAnti-EpCAM has the same binding specificity as Anti-EpCAM.

FIG. 6A Comparison of in vitro bioactivity of Anti-EpCAM and dgAnti-EpCAM using unstimulated human PBMC.

FIG. 6B Comparison of in vitro bioactivity of Anti-EpCAM and dgAnti-EpCAM using mouse splenocyte NK cells prestimulated with IL-2.

FIG. 7 Serum plasma level vs. time measured for Anti-EpCAM and dgAnti-EpCAM in a mouse model

FIG. 8 Serum peak and trough levels of Anti-EpCAM (FIG. 8A) and dgAnti-EpCAM (FIG. 8B) vs. time in a mouse model

FIG. 9 Photo of effect of Anti-EpCAM and dgAnti-EpCAM on lung tumor progression in a mouse model

FIG. 10 Graph of effect of Anti-EpCAM and dgAnti-EpCAM on lung tumor progression in a mouse model

### EXAMPLES

### General

The following examples are intended to illustrate various aspects of the invention and are in no way limiting to the invention's scope. Generally, the examples describe construction and evaluation of a domain-grafted antibody according to one embodiment of the invention starting from a fully human IgG1 antibody which binds to the human EpCAM molecule. This fully human IgG1 antibody is termed “Anti-EpCAM”, and has been previously described (Raum et al. (2001) Cancer Immunol. Immunother. 50, 141-50). The domain-grafted antibody corresponding to Anti-EpCAM is hereinafter termed “dgAnti-EpCAM”. dgAnti-EpCAM comprises VH, C_{H}1, hinge, C_{H}2, C_{H}3, VL and C_{L} regions, i.e. all regions which are also present in Anti-EpCAM. dgAnti-EpCAM differs from Anti-EpCAM in that only the VH and VL regions are of human origin (being the same as in Anti-EpCAM), whereas the C_{H}1, hinge, C_{H}2, C_{H}3, and C_{L} regions are all from the known mouse IgG2a antibody OKT3. The amino acid sequence of the heavy chain of dgAnti-EpCAM is as set out in SEQ ID NO. 2 and the amino acid sequence of the light chain of dgAnti-EpCAM is as set out in SEQ ID NO. 4.

### Example 1

### Construction of dgAnti-EpCAM

### Cell Lines

Chinese hamster ovary (CHO) dhfr-cells were obtained from the German Collection of Microorganisms and Cell Cultures (DSMZ, Braunschweig, Germany) and the KATO III human gastric carcinoma cell line from the European Collection of Cell Cultures (ECACC, Salisbury, UK). CHO dhfr-cells were grown at 37° C. in roller bottles with HyClone culture media (HyClone, Logan, Utah, USA) for 7 days before harvest. KATO III cells were cultured in RPMI 1640 media (Invitrogen, Karlsruhe, Germany), supplemented with 10% fetal bovine serum (FBS) (Invitrogen, Karlsruhe, Germany), at 37° C., in a 5% CO₂ incubator. The EpCAM transfected cell line B16F10/EpCAM (clone 3E3) was generated by Micromet. In brief, the parental cell line B16F10 was transfected with pEF-ADA-EpCAM and selected with increasing amounts of AAU (adenosine/alanosine/uridine) and dcF (desoxicoformycine). A highly EpCAM-positive clone (clone 3E3) was picked by limiting dilution analysis.

Construction and Verification of dgAnti-EpCAM

Generation and production of Anti-EpCAM has been previously described (Raum et al. (2001) Cancer Immunol. Immunother. 50, 141-50). For the generation of dgAnti-EpCAM, the constant regions were cloned by reverse transcription-PCR from RNA isolated from OKT3 hybridoma cells expressing a mouse IgG2a antibody directed against human CD3ε. For the amplification of the cH1-cH3 domains a primer (SEQ ID NO: 5) hybridizing to the 5′ end of mouse IgG2a was designed. This primer harbored a stretch of 20 nucleotides complementary to the 3′ end of the HD69 vH. The second primer (SEQ ID NO: 6) bound to the 3′ end of mouse IgG2a sequence including a stop codon and a Xba I restriction endonuclease (RE) site. For the amplification of the mouse cκ sequence a primer (SEQ ID NO: 7), which bound to the 5′ end of the mouse cκ sequence and harboured a 20-nucleotide overhang hybridizing to the Anti-EpCAM vL 3′ region, was used. The anti-sense primer (SEQ ID NO: 8) hybridized to the 3′ end of mouse cκ encoding a stop codon and a Xho I RE site. The vH of Anti-EpCAM was amplified from the expression vector pEF-DHFR HC HD69 using the primer SEQ ID NO: 9 hybridizing to the 5′ IgG signal peptide and harbouring an EcoR I RE site and the primer SEQ ID NO: 10 binding to the 3′ end of vH HD69 and having a 20 nucleotide sequence overhang complementary to the 5′ mouse IgG2a cH1 sequence. The Anti-EpCAM vL was amplified accordingly with the primers SEQ ID NO: 9 and SEQ ID NO: 11 hybridizing to the 3′ end of HD69 vL and containing on overhang binding to the 5′ end of mouse cκ. Finally, heavy and light chain sequences were generated by assembling the corresponding PCR fragments by means of overlapping PCR. For the heavy and light chain the primer combinations SEQ ID NO: 9/SEQ ID NO: 6 and SEQ ID NO: 9/SEQ ID NO: 8 were used, respectively. The complete sequence of dgAnti-EpCAM HC was then subcloned into the vector pPCR-Script-Cam, the dgAnti-EpCAM LC sequence was subcloned into pPCR-Script-Amp. The correct sequence was verified by automated sequencing. Finally, the HD69 chimeric heavy chain was cloned into the expression vector pEF-DHFR, which was digested with EcoRI and XbaI. The light chain digested with EcoRI and XhoI was inserted into pEF-ADA, which was restricted with EcoRI/SalI. dgAnti-EpCAM was produced in CHO dhfr-cells transfected with the expression vectors pEF-DHFR-HD69 HC and pEF-ADA-HD69 LC and dgAnti-EpCAM purified from cell culture supernatants in a one step process using a Protein G column and Äkta FPLC System (Amersham Biosciences, Little Chalfont, UK). The primers used above in the construction of dgAnti-EpCAM are indicated in Table 1:

TABLE 1

List of primers used in the construction
of dgAnti-EpCAM

| | |
|---|---|
| Primer code | Primer sequence* |

| | |
|---|---|
| SEQ ID NO: 5 | 5′-CCACGGTCACCGTCTCCTCAGCCAAAACAACAGCCCCATC-3′ |
| SEQ ID NO: 6 | 5′-CGTTCTAGATCATTTACCCGGAGTCCGG-3′ |
| SEQ ID NO: 7 | 5′-GGACCAAGCTGGAGCTGAAACGGGCTGATGCTGCACCAAC-3′ |
| SEQ ID NO: 8 | 5′-CCACTCGAGCCCGGGCTAACACTCATTCCTGTTGAAG-3′ |
| SEQ ID NO: 9 | 5′-AGGAATTCCACCATGGGATG-3′ |
| SEQ ID NO: 10 | 5′-GATGGGGCTGTTGTTTTGGCTGAGGAGACGGTGACCGTGG-3′ |
| SEQ ID NO: 11 | 5′-GTTGGTGCAGCATCAGCCCGTTTCAGCTCCAGCTTGGTCC-3′ |

*Primer overhangs are in italic.

Secreted dgAnti-EpCAM was purified from cell culture supernatants by Protein G affinity chromatography. SDS/PAGE and Western blot analysis indicated a purity >95% for dgAnti-EpCAM. The productivity of dgAnti-EpCAM was approximately 11 mg/l culture supernatant.

### Example 2

### Binding Comparison of Anti-EpCAM and dgAnti-EpCAM

Kinetic binding experiments with Anti-EpCAM and dgAnti-EpCAM were performed using surface plasmon resonance on the BIAcore™ 2000 (BIAcore AB, Uppsala, Sweden) with a flow rate of 5 μL/min and HBS-EP (0.01 M HEPES, pH 7.4, 0.15 M NaCl, 3 mM EDTA, 0.005% surfactant P20) as running buffer at 25° C. The extracellular domain of the EpCAM antigen (residues 17-265) was immobilized onto flow cells 2-4 on a CM5 sensor chip. The chip surface was activated injecting 80 μL of 0.1 M sodium-hydroxysuccinimide, 0.4 M N-ethyl-N′(3-dimethylaminepropyl)-carbodiimide (NHS/EDC). The antigen was coupled by manual injection of 60 μg/mL EpCAM in 0.01 M sodium acetate, pH 4.7. Different densities of antigen were immobilized on flow cells 2-4 adjusting the amount of manual injection times. Flow cell 1 was left empty while flow cell 2 was coated with the highest density of EpCAM (4100 RU). Flow cell 3 was coated with half of the amount of antigen immobilized on flow cell 2 (974 RU) and flow cell 4 was coated with lowest density of EpCAM antigen (265 RU). The activated surface of the sensor chip was blocked injecting 85 μL of 1 M ethanolamine and the chip was left to equilibrate over night at a constant flow of 5 μL/min of HBS-EP. Binding kinetics of the antibodies were measured injecting 10 μL of protein solution at concentrations ranging from 2 μM-0.07 μM and monitoring the dissociation for 100 sec. Protein was buffered in HBS-EP. The data were fitted using BIAevalution™ software determining the rate constant for dissociation and association kinetics with a 1:1 Langmuir binding equation (1, 2). A is the concentration of injected analyte and B is the concentration of (free) binding sites. At timepoint zero, B[0] is equal to the maximum Response, Rmax, meaning that at timepoint zero, all binding sites are free.

dB/dt=−(ka*[A]*[B]−kd*[AB]) (1)

dAB/dt=ka*[A]*[B]−kd*[AB] (2)

For the competition binding experiments, the binding of a single concentration of one antibody (ligand) was measured in the presence of various concentrations of the competitor antibody. In order to reach equilibrium binding, Kato III cells (50,000/well) were incubated for 16 hrs at room temperature in 50 μl of FACS buffer (PBS, 1% FCS, 0.05% NaN₃) containing the respective ligand and competitor antibody. For detection of the binding of the ligand antibody a FITC-labeled detection antibody specific for human or mouse antibodies was used (anti-human IgG-FITC, ICN 67217; anti-mouse IgG-FITC, Sigma F-6257). Assay data were analyzed with Prism software (GraphPad Software Inc.).

After nonlinear regression of the competitive binding curves the Kᵢ value for the competitor could be calculated knowing the K_{D} value from a parallel saturation binding experiment.

The plasmon resonance spectroscopy and binding competition analyses described above demonstrated that dgAnti-EpCAM retained binding affinity and specificity comparable to that of the parental human IgG1 antibody Anti-EpCAM. The equilibrium dissociation constants (K_{D}) for EpCAM binding were determined to be 66.6±33.6 nM and 90.9±36.4 nM for Anti-EpCAM and dgAnti-EpCAM, respectively. Differences in K_{D} values were not statistically relevant, indicating that affinity for EpCAM was fully maintained in dgAnti-EpCAM.

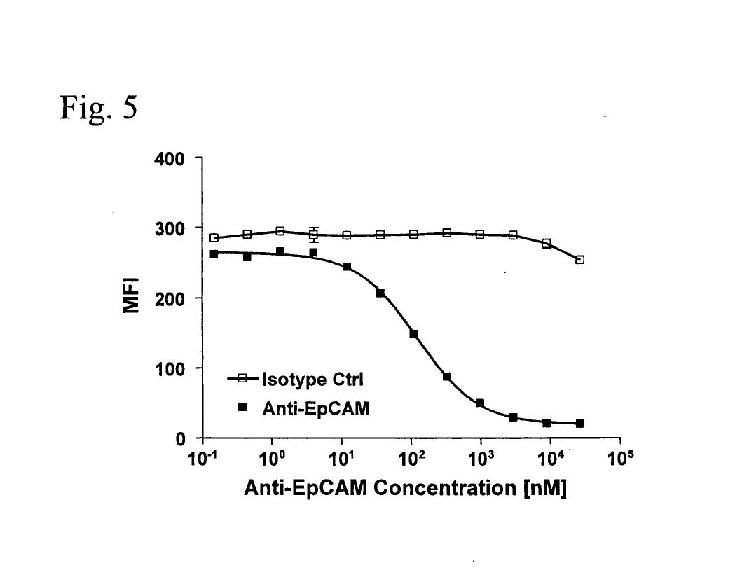

To determine whether dgAnti-EpCAM had retained the epitope specificity of Anti-EpCAM, EpCAM-expressing Kato III gastric carcinoma cells were incubated with a non-saturating concentration of fluorescently labelled dgAnti-EpCAM (4 μg/ml). In competition binding analyses increasing concentrations of Anti-EpCAM or a human IgG1 isotype control antibody of different antigen specificity were tested for displacement of the bound antibody. The results from this flow-cytometry study are shown in FIG. 5, in which the concentration of antibody is shown on the x-axis and the mean fluorescence intensity (“MFI”) is shown on the y-axis. Here, a decrease in MFI may be taken as an indication that labelled dgAnti-EpCAM has been competitively displaced by Anti-EpCAM from the surface of the EpCAM-bearing Kato III cells. Such a decrease may therefore be taken to mean that Anti-EpCAM and dgAnti-EpCAM bound to the same epitope of the same antigen. As is clearly visible from FIG. 5, increasing concentrations of Anti-EpCAM (filled squares) led to an increasing attenuation of the MFI, and therefore to an increasing displacement of dgAnti-EpCAM from its EpCAM antigen. In contrast, another antibody of the same isotype but another antigen binding specificity (open squares, “isotype control”) did not displace dgAnti-EpCAM from its antigen. Anti-EpCAM therefore effectively competed with dgAnti-EpCAM for binding to Kato III cells while the isotype control antibody had no effect, meaning that antigen binding specificity was retained in the conversion of Anti-EpCAM to dgAnti-EpCAM.

### Example 3

### Bioactivity Comparison of Anti-EpCAM and dgAnti-EpCAM

Following the determination that both binding affinity and binding specificity were retained in the conversion of Anti-EpCAM to dgAnti-EpCAM, it was then desired to establish whether bioactivity had also been retained. In order to determine this, ADCC assays were performed. Briefly, in an ADCC assay, one employs two kinds of cells: cells expressing the human cell-surface molecule specifically bound by the antibody to be tested (these are the “target cells”) and cells which are capable of killing target cells which have become decorated with antibody (these killing cells are the “effector cells”). As target cells will be lysed by effector cells via ADCC, the degree of ADCC can be quantified by quantifying the amount of target cell lysis. Generally, this quantification is expressed as an EC₅₀ value, referring to the concentration of antibody required to effect half-maximal cell lysis. A lower EC₅₀ value thus is indicative of higher potency.

For ADCC assays, murine NK cells (the effector cells) were prepared by negative selection of C57BL/6 splenocytes using the murine NK cell isolation kit from BD Biosciences (San Jose, Calif., USA) as described by the manufacturer. Isolated NK cells were cultured for 7-14 days in RPMI 1640/10% FCS supplemented with 1700 U/ml Proleukin (Chiron GmbH, Munich, Germany) at a density of about 1×10⁶ cells/ml. Every 2-3 days cells were counted and fresh medium added. After 7 to 14 days in culture, NK cell purity was approximately 90 to 100%. Stimulated murine NK cells were resuspended in RPMI 1640/10% FCS at a concentration of 1.6×10⁷ cells/ml and used as effector cells in ADCC assays. For the preparation of human effector cells peripheral blood mononuclear cells (“PBMC”) were enriched by Ficoll-Hypaque gradient centrifugation (Naundorf et al), washed and re-suspended at 1.2×10⁷/ml.

EpCAM-positive Kato III cells were used as target cells and were labeled with the fluorescent membrane dye PKH-26 (Sigma, Taufkirchen, Germany) according to the manufacturer's protocol to distinguish target from effector cells in the FACS analysis. PKH-26-labeled target cells were adjusted to a density of 4×10⁵ cells/ml and 6×10⁵ cells/ml for assays with murine and human effector cells, respectively. Equal volumes of target and effector cell suspensions were mixed, resulting in an effector-to-target (E:T) ratio of approximately 50:1 and 20:1 for murine and human effector cells, respectively, and 50 μl were added per well of a 96-well U-bottom microtiter plate (Greiner, Solingen, Germany). Four-fold serial dilutions of Anti-EpCAM and 10-fold serial of dgAnti-EpCAM were prepared and 50 μl were added per well resulting in a concentration range of 50,000-0.05 ng/ml for Anti-EpCAM and 50,000-0.2 ng/ml for dgAnti-EpCAM. ADCC reactions were incubated for 10 and 4 hours at 37° C. for assays with murine and human effector cells, respectively. Propidium iodide (PI) was added to a final concentration of 1 μg/ml and 5×10⁴ cells analyzed by flow cytometry using a FACSCalibur (Becton Dickinson, Heidelberg, Germany). Dose response curves were computed by nonlinear regression analysis using a four-parameter-fit-model provided with the GraphPad Prism software package (GraphPad Software, San Diego, Calif., USA). All experiments were performed in triplicate. Quantification of cytotoxicity was based on the number of dead target cells in relation to the total number of target cells in each test sample. The specific cytotoxicity was calculated by the formula: [dead target cells (sample)/total target cells (sample)]×100.

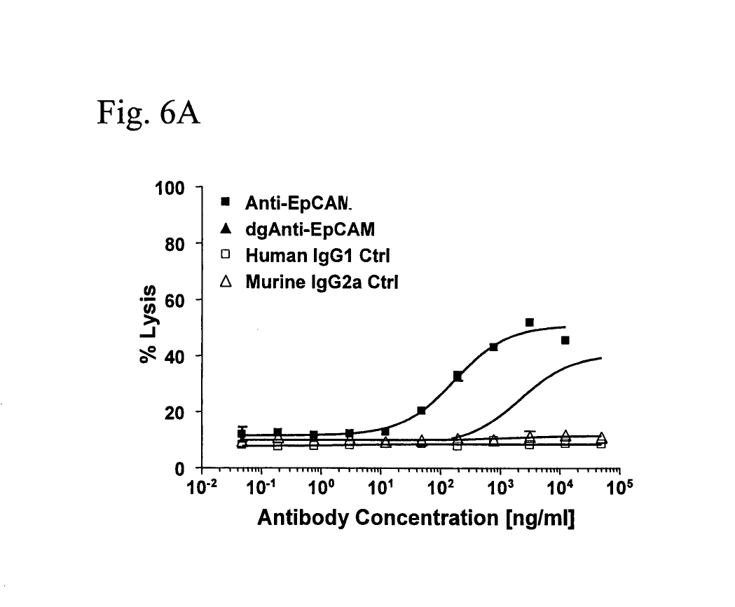

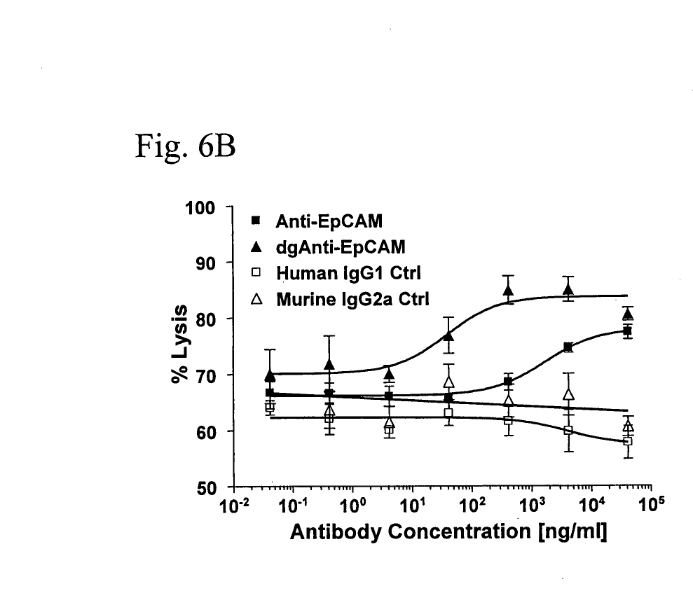

The results of this experiment are shown in FIG. 6. As can be seen in FIG. 6A, Anti-EpCAM showed a much higher ADCC activity than dgAnti-EpCAM when unstimulated human PBMC were used as effector cells. This is apparent due to the lower EC₅₀ value determined for Anti-EpCAM than for dgAnti-EpCAM. EC₅₀ was seen at a concentration of 169.6 ng/ml for Anti-EpCAM versus 2,110 ng/ml for dgAnti-EpCAM, resulting in a 12.4-fold potency difference. It is known that human PBMC are capable of ADCC effector function without prior stimulation (i.e. in an unstimulated state), whereas murine effector cells such as murine NK cells require prestimulation, for example with IL-2, in order to elicit ADCC (Niwa et al. (2004) Cancer Research 64, 2127-33). In standing with this, neither Anti-EpCAM nor dgAnti-EpCAM elicited ADCC activity when tested in assays with unstimulated mouse splenocytes or NK cells isolated therefrom (data not shown). While Anti-EpCAM did elicit dose-dependent ADCC activity with murine effector (NK) cells prestimulated with IL-2 employed at an E:T ratio of 50:1, dgAnti-EpCAM was found to be more efficacious in this regard (FIG. 6B). Specifically, dgAnti-EpCAM induced half-maximal target cell lysis at a concentration of 38.1 ng/ml and Anti-EpCAM at 1,664 ng/ml, resulting in 43.7-fold higher potency of dgAnti-EpCAM when using prestimulated murine NK cells as effector cells. Human IgG1 and murine IgG2a isotype control antibodies did not elicit ADCC activity under either experimental condition, i.e. using human or murine effector cells, underscoring the target specificity of Anti-EpCAM and dgAnti-EpCAM.

Taken together, FIGS. 6A and 6B support the notion that an antibody's capacity to elicit ADCC will be most completely realized when this antibody's Fc portion originates from the same species as the organism in which ADCC is elicited. Seen another way, the most accurate indication of the propensity of an antibody to trigger ADCC may be obtained using an antibody in which the origin of the Fc portion correlates to the species used for testing ADCC.

### Example 4

### Pharmacokinetic Comparison of Anti-EpCAM and dgAnti-EpCAM in Mice

### Animal Studies

In-vivo experiments were performed in female 6-10 week old immunocompetent C57BL/6 mice bred at the Institute of Immunology (Munich, Germany). The mice were maintained under sterile and standardized environmental conditions (20±1° C. room temperature, 50±10% relative humidity, 12-h light-dark-rhythm) and received autoclaved food and bedding (ssniff, Soest, Germany) as well as acidified (pH 4.0) drinking water ad libitum. All experiments were performed according to the German Animal Protection Law with permission from the responsible local authorities. Statistical analysis of the mean number of lung tumor colonies of the corresponding treatment groups versus the vehicle control group was performed using the Student's t-test.

### Pharmacokinetic Analysis

It was then desired to generate a pharmacokinetic profile of Anti-EpCAM and dgAnti-EpCAM. To this end 20 female C57BL/6 mice were intravenously injected with 300 μg of the respective antibody. Animals were allocated to 4 different groups of 5 mice each. Different groups were alternatingly bled at different time points after injection (predose, 0.5, 1, 2, 4 and 10 hrs, 1, 2, 4, 7, 9, 11, 14, 18, 21, 24 and 28 days). Serum concentrations quantified by specific ELISAs. ELISA plates (NUNC, Wiesbaden, Germany) were coated with 100 μl (5 μg/mL) of rat anti-Anti-EpCAM antibody (Micromet AG, Munich, Germany). Plates were incubated overnight at 4° C. and blocked with PBS/1% bovine serum albumin (BSA) for 60 min at 25° C. Test samples were diluted in PBS/10% mouse plasma pool, 100 μl added per well and incubated for 60 min at 25° C. For Anti-EpCAM quantification, plates were incubated with 100 μl (0.15 μg/mL) of chicken anti-Anti-EpCAM antibody conjugated with biotin (Micromet, Munich, Germany) at a final concentration of 2 μg/ml for 60 min at 25° C. followed by incubation for 60 min at 25° C. with 100 μl streptavidin conjugated with alkaline phosphatase (Dako, Hamburg, Germany) at a final concentration of 0.5 μg/ml. For dgAnti-EpCAM quantification plates were incubated with 100 μl of goat anti-mouse antibody conjugated with alkaline phosphatase (Sigma, Taufkirchen, Germany) for 60 min at 25° C. Finally, plates were incubated with 100 μl of substrate (1 mg/ml of p-NPP dissolved in 0.2 M TRIS buffer; Sigma, Taufkirchen, Germany) for 20 minutes at 25° C. and the absorbance (405 nm) read on Power WaveX select (Bio-Tek instruments, USA). Two-fold serial dilutions of each test sample were analyzed in duplicate and OD values that were within the linear range of the standard curve were used to calculate the concentration of Anti-EpCAM and dgAnti-EpCAM. Pharmacokinetic calculations of Anti-EpCAM and dgAnti-EpCAM were performed by the pharmacokinetic software package WinNonlin Professional 4.1 (Pharsight Corporation, Mountain View, Calif.; 2003). Parameters were determined by non-compartmental analysis (NCA). The non-compartmental analysis was based on model 201 (intravenous bolus injection).

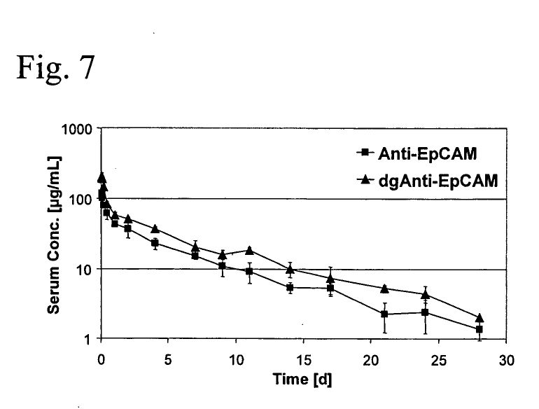

Single administration of 300 μg Anti-EpCAM and dgAnti-EpCAM resulted in maximum serum concentrations (Cₘₐₓ) of 119.2 μg/ml and 204 μg/ml, respectively, 30 min after i.v. bolus injection into C57BL/6 mice. Serum concentrations of the antibodies were well detectable until the end of the 28-day study period as shown in FIG. 7. Serum concentration versus time profiles for both Anti-EpCAM and dgAnti-EpCAM exhibited a bi-exponential curve progression with an early distribution phase between 0 and 10 hours and a terminal elimination phase. Despite curve progression looking similar for both antibodies, dgAnti-EpCAM doses resulted in constantly higher serum concentration compared to Anti-EpCAM, which was also reflected by higher exposure (AUCₗₐₛₜ) values of 519.8 day*μg/ml for dgAnti-EpCAM versus 335.9 day*μg/ml for Anti-EpCAM. The volume of distribution (Vz) and the clearance (CL) were calculated to be 5.28 ml and 0.56 ml/hr for dgAnti-EpCAM, and with 7.78 ml and 0.86 ml/hr for Anti-EpCAM. Both the volume of distribution and the clearance were higher for Anti-EpCAM as compared to dgAnti-EpCAM. The elimination rate constants resulted in similar distribution half-lives (T_{1/2-alpha}) of 0.27 and 0.31 days and terminal elimination half-lives (T_{1/2-beta}) of 6.21 and 6.57 days for Anti-EpCAM and dgAnti-EpCAM, respectively.

The results shown in FIG. 7 show that Anti-EpCAM is cleared from mouse test animals more rapidly than dgAnti-EpCAM. In an attempt to compensate for this pharmacokinetic disparity, further studies in test mice were performed by administering a higher level of Anti-EpCAM than dgAnti-EpCAM such that the serum peak and trough levels for both antibodies in mice would remain as identical, and therefore the results as comparable, as possible.

### Example 5

### Animal Tumor Models

Finally, it was desired to test Anti-EpCAM and dgAnti-EpCAM in actual animals. To this end, Anti-EpCAM and dgAnti-EpCAM were compared side-by-side in immunocompetent C57BL/6 mice. One ×10⁵ B16/EpCAM cells (forming tumor cell colonies in the lung) were intravenously injected into C57BL/6 mice and animals treated 3-times a week with the indicated dose levels of Anti-EpCAM, dgAnti-EpCAM or human IgG control antibody starting one hour after B16/EpCAM inoculation. In order to render B16F10 cells suitable for the immunotherapy with the EpCAM-specific antibodies, the cells were transfected with an expression vector encoding human EpCAM. The subclone B16/EpCAM 3E3 stably expressing human EpCAM, was selected and the EpCAM expression determined by saturation binding. Approximately 2.0×10⁶ EpCAM binding sites were measured. This number is comparable to the 1.3×10⁶ EpCAM sites expressed on KATO III cells. The high-level of EpCAM expression on B16/EpCAM cell was found to be stable for at least 6 weeks in cell culture, even in the absence of a selection pressure (data not shown), assuring stable EpCAM expression on tumor cells during efficacy studies in mice.

Mice were sacrificed and dissected on day 26 after B16/EpCAM injection. Lungs were filled with tissue teck (Vogel GmbH, Giessen, Germany) and analyzed macroscopically for the number of tumor colonies. To monitor exposure to the respective antibodies three animals per group were alternately bled before and 30 minutes after the 3^{th}, 6^{th}, 9^{th}, 11^{th} infusion as well as at the end of the study.

In an establishment phase, the number of cells to be injected and time to read-out were defined. The syngeneic B16/EpCAM cells were intravenously injected into C57BL/6 mice and the number of tumor colonies in lung tissue were counted at different time points after inoculation. Conditions under which 1×10⁵ injected B16/EpCAM cells resulted in an average of 80-100 tumor colonies between 21 and 28 days after injection were chosen for the efficacy studies.

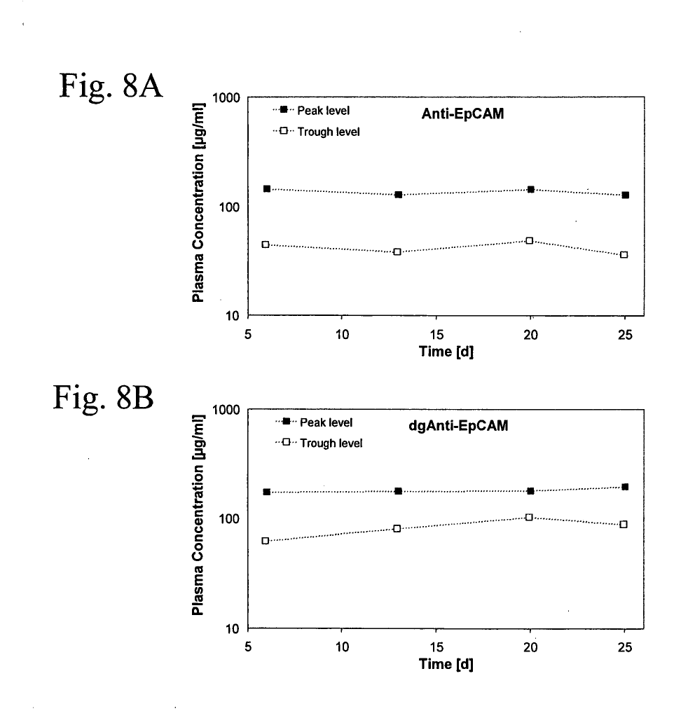

The single-dose pharmacokinetic profiles of Anti-EpCAM and dgAnti-EpCAM were used for modelling a dosing regimen that would result in serum trough levels at or above 30 μg/ml, the targeted trough levels of Anti-EpCAM in two ongoing clinical phase II studies. Based on modelling, a loading dose of 600 μg/mouse followed by maintenance doses of 250 μg/mouse 3-times per week were selected for Anti-EpCAM and for the human IgG control antibody. For dgAnti-EpCAM, a loading dose of 300 μg/mouse and maintenance doses of 125 μg/mouse 3 times a week were administered. Following intravenous inoculation with 1×10⁵ B16/EpCAM, ten animals per group were treated with the antibodies and serum levels of Anti-EpCAM (FIG. 8A) and dgAnti-EpCAM (FIG. 8B) determined after the 3^{rd}, 6^{th}, 9^{th} and 11^{th} administration. Anti-EpCAM injections resulted in mean peak to trough plasma concentrations of 136 to 41 μg/ml and were close to the expected plasma concentrations of 150 to 30 μg/ml during the course of the study. Mean peak to trough concentration of dgAnti-EpCAM were determined with 172 to 82 μg/ml. Although plasma concentrations of dgAnti-EpCAM were slightly higher than for Anti-EpCAM, the overall exposure with both antibodies was considered to be in an effective and comparable range.

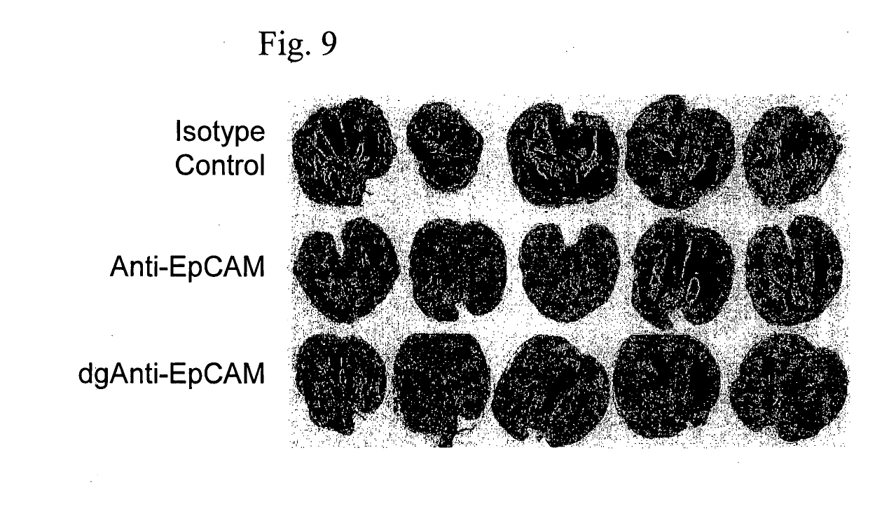

Macroscopic inspection of mouse lungs showed that both the human and murinized anti-EpCAM antibody led to a strong reduction of tumor growth compared to the isotype control (FIG. 9). While lungs from mice treated with dgAnti-EpCAM had very few detectable tumors (bottom panels), tiny tumors were still visible on lungs from mice treated with the human Anti-EpCAM (middle panels). Although the size of the tumor colonies was smaller than the size in lungs of animals treated with the human IgG1 isotype, the number of colonies was only slightly reduced after Anti-EpCAM treatment. In contrast, treatment with dgAnti-EpCAM induced a highly significant reduction in the number of lung tumor colonies by >85% (p<0.0001), and the few remaining tumor colonies were of very small size.

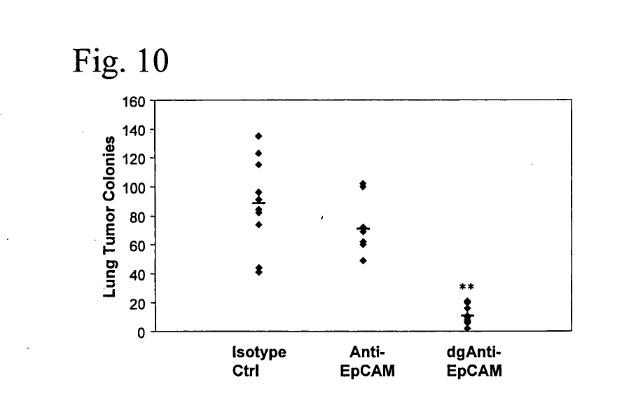

The pictorial results shown in FIG. 9 are represented in graphical form in FIG. 10. Here the clearly higher cytotoxic activity of dgAnti-EpCAM in mice as compared to Anti-EpCAM is evident in the low number of remaining lung tumor colonies in lungs belonging to mice treated with dgAnti-EpCAM.

1. A domain-grafted antibody which specifically binds a human cell-surface molecule, said domain-grafted antibody comprising
(a) an antibody heavy chain variable region of human origin;
(b) an antibody light chain variable region of human origin;
(c) a second antibody heavy chain constant region (C_{H}2) from a non-human species; and
(d) an antibody heavy chain hinge region from said non-human species;
said antibody heavy and light chain variable regions together defining a binding site for said human cell-surface molecule.

2. The domain-grafted antibody of claim 1, further comprising a third antibody heavy chain constant region (C_{H}3) from said non-human species.

3. The domain-grafted antibody of claim 1, further comprising a first antibody heavy chain constant region (C_{H}1) from said non-human species and an antibody light chain constant region (C_{L}) from said non-human species.

4. The domain-grafted antibody of claim 1, wherein the antibody light chain constant region (C_{L}) is a kappa antibody light chain constant region.

5. The domain-grafted antibody of any of claim 1, wherein the antibody heavy and light chain variable regions of human origin are independently human or humanized.

6. The domain-grafted antibody of claim 1, wherein said non-human species is a rodent species, a non-human primate species, rabbit, beagle dog, pig, mini-pig, goat or sheep.

7. The domain-grafted antibody of claim 6, wherein said non-human primate species is chimpanzee, cynomolgous monkey, rhesus monkey, baboon or marmoset.

8. The domain-grafted antibody of claim 6, wherein the rodent species is mouse, rat, guinea pig, hamster or gerbil.

9. The domain-grafted antibody of claim 8, wherein the rodent species is mouse and the antibody first heavy chain constant region, the second antibody heavy chain constant region, the third antibody heavy chain constant region and the antibody heavy chain hinge region are of the gamma isotype.

10. The domain-grafted antibody of claim 9, wherein the subclass of the gamma isotype is chosen from the group consisting of gamma 1, gamma 2a, gamma 2b and gamma 3.

11. The domain-grafted antibody of claim 1, wherein said human cell-surface molecule is exclusively expressed or overexpressed in a pathological state or is more readily accessible for antibody binding in a pathological state than in a non-pathological state.

12. The domain-grafted antibody of claim 11, wherein the human cell-surface molecule is present in a pathological state and the pathological state is a proliferative disease, especially a tumorous disease.

13. The domain-grafted antibody of claim 12, wherein the human cell-surface molecule is present in a tumorous disease and the tumorous disease is a cancerous.

14. The domain-grafted antibody of claim 13, wherein the human cell-surface molecule is human EpCAM.

15. The domain-grafted antibody of claim 11, wherein the human cell-surface molecule is present in a pathological state and the pathological state is a pathogen-related disease.

16. The domain-grafted antibody of claim 15, wherein the pathogen-related disease is a viral disease or a retroviral disease.

17. The domain-grafted antibody of claim 16, wherein the viral disease is caused by herpes simplex virus (HSV), human papilloma virus (HPV), cytomegalovirus (CMV) or Epstein-Barr Virus (EBV).

18. The domain-grafted antibody of claim 16, wherein the retroviral disease is caused by human immunodeficiency virus (HIV).

19. The domain-grafted antibody of claim 11, wherein the human cell-surface molecule is present in a pathological state and the pathological state is an inflammatory disease.

20. The domain-grafted antibody of claim 19, wherein the human cell-surface molecule is human membrane-bound IgE.

21. The domain-grafted antibody of claim 19, wherein the human cell-surface molecule is a human chemokine receptor, a human cytokine receptor or a human c-type lectin receptor.

22. The domain-grafted antibody of claim 21, wherein the human cytokine receptor is the human granulocyte-macrophage colony stimulating factor (GM-CSF) receptor or human CCR5.

23. The domain-grafted antibody of claim 21, wherein the human c-type lectin receptor is human NKG2D.

24. An expression vector, said expression vector comprising:
(i) a first coding sequence encoding: a) a heavy chain variable region of human origin, (b) a second antibody heavy chain constant region (CH2) from a non-human species and (c) an antibody heavy chain hinge region from said non-human species, and optionally, (d) a third antibody heavy chain constant region (CH3) from said non-human species and/or (e) a first antibody heavy chain constant region (CH1) from said non-human species and/or;
(ii) a second coding sequence encoding: a desired antibody light chain variable region (VL) of human origin and, optionally, an antibody light chain constant region (CL) from said non-human species;
said antibody heavy and light chain variable regions together defining a binding site for a human cell-surface molecule.

25. A host cell comprising the expression vector of claim 24.

26. A method of producing a domain-grafted antibody comprising culturing a host cell under conditions suitable for growth of said host cell, said host cell comprising an expression vector, said expression vector comprising:
(i) a first coding sequence encoding: a) a heavy chain variable region of human origin (b) a second antibody heavy chain constant region (CH2) from a non-human species and (c) an antibody heavy chain hinge region from said non-human species, and optionally, (d) a third antibody heavy chain constant region (CH3) from said non-human species and/or (e) a first antibody heavy chain constant region (CH1) from said non-human species and/or;
(ii) a second coding sequence encoding: a desired antibody light chain variable region (VL) of human origin and, optionally, an antibody light chain constant region (CL) from said non-human species;
said antibody heavy and light chain variable regions together defining a binding site for a human cell-surface molecule.

27. The method of claim 26, said culturing being performed in serum-free medium.

28. The method of claim 26, further comprising isolating said domain-grafted antibody.

29. The method of claim 28, further comprising purifying said domain-grafted antibody.

30. The method of claim 29, further comprising formulating said domain-grafted antibody into a pharmaceutical composition.

31. A pharmaceutical composition comprising a domain-grafted antibody according to claim 1.

32. A method of measuring the in vivo activity of a pharmaceutical composition comprising the domain-grafted antibody according to claim 1, the method comprising administering the composition or said domain-grafted antibody to a non-human animal expressing a human cell-surface molecule and measuring said in vivo activity of said composition or said domain-grafted antibody, wherein at least the second antibody heavy chain constant region (C_{H}2) and the antibody heavy chain hinge region of said domain-grafted antibody are from the same species of non-human animal as the non-human animal to which said domain-grafted antibody or composition is administered.

33. The method of claim 32, wherein the non-human animal is a transgenic non-human animal.

34. The method of claim 32, wherein the non-human animal is of rodent species, of non-human primate species, a rabbit, a beagle dog, a pig, a mini-pig, a goat or a sheep.

35. The method of claim 31, wherein the animal of rodent species is a mouse, a rat, a guinea pig, a hamster or a gerbil.

36. The method of claim 34, wherein the animal of non-human primate species is a chimpanzee, a cynomolgous monkey, a rhesus monkey, a baboon or a marmoset.

37. (canceled)

38. The method of claim 32, wherein the in vivo activity is in vivo cytotoxicity.

39. The method of claim 26, wherein said host cell is a mammalian cell.

40. The method of claim 39, wherein said mammalian cell is a CHO cell.


SEQUENCE LISTING (11 sequences)

SEQ ID NO: 1 - DNA, 1520 bp, artificial sequence
  dgAnti-EpCAM heavy chain (nucleotide)
gaattccaccatgggatggagctgtatcatcctcttcttggtagcaacagctacaggtaaggggctcacagtagcaggcttgaggtctggacatatatatgggtgacaatgacatccactttgcctttctctccttaggtgtccactccgaggtgcagctgctcgagtctgggggaggcgtggtccagcctgggaggtccctgagactctcctgtgcagcctctggattcaccttcagtagctatggcatgcactgggtccgccaggctccaggcaaggggctggagtgggtggcagttatatcatatgatggaagtaataaatactatgcagactccgtgaagggccgattcaccatctccagagacaattccaagaacacgctgtatctgcaaatgaacagcctgagagctgaggacacggctgtgtattactgtgcgaaagatatggggtggggcagtggctggagaccctactactactacggtatggacgtctggggccaagggaccacggtcaccgtctcctcagccaaaacaacagccccatcggtctatccactggcccctgtgtgtggagatacaactggctcctcggtgactctaggatgcctggtcaagggttatttccctgagccagtgaccttgacctggaactctggatccctgtccagtggtgtgcacaccttcccagctgtcctgcagtctgacctctacaccctcagcagctcagtgactgtaacctcgagcacctggcccagccagtccatcacctgcaatgtggcccacccggcaagcagcaccaaggtggacaagaaagagcccagagggcccacaatcaagccctgtcctccatgcaaatgcccagcacctaacctcttgggtggaccatccgtcttcatcttccctccaaagatcaaggatgtactcatgatctccctgagccccatagtcacatgtgtggtggtggatgtgagcgaggatgacccagatgtccagatcagctggtttgtgaacaacgtggaagtacacacagctcagacacaaacccatagagaggattacaacagtactctccgggtggtcagtgccctccccatccagcaccaggactggatgagtggcaaggagttcaaatgcaaggtcaacaacaaagacctcccagcgcccatcgagagaaccatctcaaaacccaaagggtcagtaagagctccacaggtatatgtcttgcctccaccagaagaagagatgactaagaaacaggtcactctgacctgcatggtcacagacttcatgcctgaagacatttacgtggagtggaccaacaacgggaaaacagagctaaactacaagaacactgaaccagtcctggactctgatggttcttacttcatgtacagcaagctgagagtggaaaagaagaactgggtggaaagaaatagctactcctgttcagtggtccacgagggtctgcacaatcaccacacgactaagagcttctcccggactccgggtaaatga

SEQ ID NO: 2 - protein, 456 aa, artificial sequence
  dgAnti-EpCAM heavy chain (amino acid)
EVQLLESGGGVVQPGRSLRLSCAASGFTFSSYGMHWVRQAPGKGLEWVAVISYDGSNKYYADSVKGRFTISRDNSKNTLYLQMNSLRAEDTAVYYCAKDMGWGSGWRPYYYYGMDVWGQGTTVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 3 - DNA, 907 bp, artificial sequence
  dgAnti-EpCAM light chain (nucleotide)
gaattccaccatgggatggagctgtatcatcctcttcttggtagcaacagctacaggtaaggggctcacagtagcaggcttgaggtctggacatatatatgggtgacaatgacatccactttgcctttctctccttaggtgtccactccgagctccagatgacccagtctccatcctccctgtctgcttctgtgggagacagagtcaccatcacttgtcggacaagtcagagcattagcagctatttaaattggtatcagcagaaaccaggacagcctcctaagctgctcatttactgggcatctacccgggaatccggggtccctgaccgattcagtggcagcgggtctgggacagatttcactctcaccatcagcagtctacaacctgaagattctgcaacttactactgtcagcagagttacgacatcccgtacacttttggccaggggaccaagctggagatcaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccatcgtcaagagcttcaacaggaatgagtgttagagacaaaggtcctgagatgccaccaccagctccccagctccatcctatcttcccttctaaggtcttggaggcttccccacaagcgacctaccactgttgcggtgctccaaacc

SEQ ID NO: 4 - protein, 214 aa, artificial sequence
  dgAnti-EpCAM light chain (amino acid)
ELQMTQSPSSLSASVGDRVTITCRTSQSISSYLNWYQQKPGQPPKLLIYWASTRESGVPDRFSGSGSGTDFTLTISSLQPEDSATYYCQQSYDIPYTFGQGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 5 - DNA, 40 bp, artificial sequence
  primer sequence
ccacggtcaccgtctcctcagccaaaacaacagccccatc

SEQ ID NO: 6 - DNA, 28 bp, artificial sequence
  primer sequence
cgttctagatcatttacccggagtccgg

SEQ ID NO: 7 - DNA, 40 bp, artificial sequence
  primer sequence
ggaccaagctggagctgaaacgggctgatgctgcaccaac

SEQ ID NO: 8 - DNA, 37 bp, artificial sequence
  primer sequence
ccactcgagcccgggctaacactcattcctgttgaag

SEQ ID NO: 9 - DNA, 20 bp, artificial sequence
  primer sequence
aggaattccaccatgggatg

SEQ ID NO: 10 - DNA, 40 bp, artificial sequence
  primer sequence
gatggggctgttgttttggctgaggagacggtgaccgtgg

SEQ ID NO: 11 - DNA, 40 bp, artificial sequence
  primer sequence
gttggtgcagcatcagcccgtttcagctccagcttggtcc